SEQUENCE LISTING (13 sequences)

SEQ ID NO: 1 - protein, 25 aa, Mus musculus
ASRNKANDYTTDYSASVKGRFIVSR

SEQ ID NO: 2 - protein, 17 aa, Mus musculus
KGEGAAVLLPVLLAAPG

SEQ ID NO: 3 - protein, 28 aa, Artificial Sequence
  T15 autophilic peptide - scrambled
SYSASRFRKNGSIRAVEATTDVNSAYAK

SEQ ID NO: 4 - protein, 24 aa, Artificial Sequence
  T15 autophilic peptide - scrambled
SKAVSRFNAKGIRYSETNVDTYAS

SEQ ID NO: 5 - protein, 17 aa, Artificial Sequence
  R24 autophilic peptide
GAAVAYISSGGSSINYA

SEQ ID NO: 6 - protein, 18 aa, Artificial Sequence
  R24 - charged autophilic peptide
GKAVAYISSGGSSINYAE

SEQ ID NO: 7 - protein, 18 aa, Artificial Sequence
  membrane transporter sequence optimized peptide
WKGESAAVILPVLIASPG

SEQ ID NO: 8 - protein, 5 aa, Artificial Sequence
  synthetic peptide with terminal tryptophan
KAAGW

SEQ ID NO: 9 - protein, 12 aa, Artificial Sequence
  synthetic peptide with terminal tryptophan
KAAKGEAKAAGW

SEQ ID NO: 10 - protein, 56 aa, Artificial Sequence
  T15 autophilic dipeptide
ASRNKANDYTTDYSASVKGRFIVSGGGRRGGGASRNKANDYTTDYSASVKGRFIVS

SEQ ID NO: 11 - protein, 51 aa, Artificial Sequence
  T15 tandem autophilic peptide
ASRNKANDYTTDYSASVKGRFIVSGGGASRNKANDYTTDYSASVKGRFIVS

SEQ ID NO: 12 - protein, 42 aa, Artificial Sequence
  membrane transporter sequence dipeptide
KGEGAAVLLPVLLAAPGGGGRRGGGKGEGAAVLLPVLLAAPG

SEQ ID NO: 13 - protein, 37 aa, Artificial Sequence
  membrane transporter sequence tandem
KGEGAAVLLPVLLAAPGGGGKGEGAAVLLPVLLAAPG

(19) United States
(12) Patent Application Publication
(10) Pub. No.: US 2009/0208418 A1
(43) Pub. Date: Aug. 20, 2009

(54) SUPERANTIBODY SYNTHESIS AND USE IN DETECTION, PREVENTION AND TREATMENT OF DISEASE

(75) Inventors: Heinz Kohler, Lexington, KY (US); Sybille Muller, Lexington, KY (US); Alton C. Morgan Jr., Scottsdale, AZ (US)

Correspondence Address: GIFFORD, KRASS, SPRINKLE,ANDERSON & CITKOWSKI, P.C, PO BOX 7021, TROY, MI, 48007-7021, US

(73) Assignee: InNexus Biotechnology Internaltional Ltd., Scottsdale, AZ (US)

(21) Appl. No.: 11/912,992

(22) PCT Filed: Apr. 29, 2005
(86) PCT No.: PCT/US2006/016844

§ 371 Date: Jan. 28, 2008

Related U.S. Application Data
(63) Continuation-in-part of application No. 11/119,404, filed on Apr. 29, 2005.

(51) Int. Cl. A61K 49/00; C07K 16/18; C12N 9/96; A61K 39/395; G01N 33/53

(52) U.S. Cl. 424/9.6; 530/391.3; 435/188; 424/178.1; 435/7.2

(57) ABSTRACT

Superantibodies having enhanced autophilic, catalytic, and/or membrane-penetrating properties are prepared by affinity-based conjugation of a photoactivatable organic molecule to a target immunoglobulin. The photoactivatable organic molecule bears a chromophoric aromatic hydrocarbon moiety, which has affinity for the immunoglobulin. Upon photolysis, the organic molecule is covalently linked to the immunoglobulin. A preferred organic molecule is a peptide and a preferred aromatic hydrocarbon moiety is a tryptophan residue. The photoactivatable organic molecule need not bear a purine, pyrimidine or azido group to effect binding to the immunoglobulin and/or photoactivation. The superantibodies can enhance the potency and expand the targeting range of target antibodies. Autophilic superantibodies can promote apoptosis of target cells and/or enhance therapeutic efficacies in the treatment of patients with diseases or disorders responsive to antibody therapy. Exemplary of such diseases are atherosclerosis and cardiovascular disease. Membrane-penetrating superantibodies can prevent apoptosis by binding to intracellular anti-caspase signal proteins. Compositions containing the superantibodies, as well as methods of making and using them, are disclosed.

### REFERENCE TO RELATED APPLICATIONS

This application is a continuation-in-part of U.S. patent application Ser. No. 11/119,404, filed 29 Apr. 2005, and is a continuation-in-part of U.S. patent application Ser. No. 10/652,864, filed 29 Aug. 2003, which claims the benefit of U.S. Provisional Patent Application No. 60/407,421, filed 30 Aug. 2002, and is a continuation-in-part of U.S. patent application Ser. No. 09/865,281, filed 29 May 2001, which is a continuation-in-part of U.S. patent application Ser. No. 09/070,907, filed 4 May 1998, now U.S. Pat. No. 6,238,667. The disclosures of the aforementioned applications are incorporated herein by reference in their entireties.

### TECHNICAL FIELD OF THE INVENTION

The present invention relates to antibodies, methods of making the same, and methods of using the antibodies in the detection, prevention, and/or treatment of a variety of disease conditions.

### BACKGROUND OF THE INVENTION

Antibodies have emerged as a major therapeutic tool for the treatment of chronic diseases, such as cancer and autoimmune disorders. Notable success stories include Herceptin® in the treatment of breast cancer and Rituxan® in the treatment of non-Hodgkin's lymphoma. A key advantage of antibodies in the treatment of disease lies in their ability to target disease-causing cells or molecules, while sparing healthy tissues and normal products of the body. However, antibodies that exhibit desired specificities in laboratory studies often fail in pre-clinical and clinical evaluations because of inefficient targeting, low therapeutic efficacy, and/or unacceptable side effects.

It is known that a major mechanism by which therapeutic antibodies are effective against their target cells is by inducing cell death, i.e., antibody-induced apoptosis. Such induced apoptosis is typically triggered by crosslinking receptors that are part of the cell's apoptosis signal pathway. For example, crosslinking the B-cell antigen receptor by means of antibodies induces apoptosis in B-cell tumors (Ghetie M., et al., 1997). Crosslinking of cellular receptors also increases the binding avidity of an antibody to its target antigen, and thus is likely to increase all cell surface-dependent therapeutic mechanisms, such as complement-mediated killing and complement-dependent opsonization and phagocytosis, antibody-dependent cellular cytotoxicity (ADCC), as well as enhanced inhibition of cell growth or alterations in metabolic pathways within cells through increased binding to and blockade of cellular receptors when using antibodies targeted to cellular receptors.

A rare class of self-binding antibodies, variously known as “autophilic antibodies” or “autobodies”, has been identified in Nature. They are capable of forming dimers and/or polymers through noncovalent interactions with self. One example of an autophilic antibody is TEPC-15, which targets a normally cryptic determinant of phosphorylcholine on apoptotic cells and atherosclerotic lesions (Binder, J., et al., 2003; Kang, C-Y, et al., 1988). Dimerization or multimerization may be induced only after the modified antibody attaches to its cell surface target, i.e., “differential oligomerization”. In solution, an autophilic antibody can be in equilibrium between its monomeric and dimeric forms (Kaveri S., et al., 1990).

Autophilic antibodies belong to a larger class of antibodies, referred to herein as “SuperAntibodies™” Super-antibodies, as used herein, exhibit one or more beneficial properties in addition to the antigen binding properties usually associated with antibodies (Kohler H., et al., 1998; Kohler H., 2000). Specifically, the referenced class of super-antibodies comprises antibodies having catalytic, adjuvant, membrane-penetrating, and/or autophilic properties, and includes molecules that afford superior targeting and therapeutic properties. Such super-antibodies are considered chimeric and typically comprise an antibody or antibody fragment covalently linked to at least one non-antibody moiety, such as a peptide, which has catalytic, adjuvant, membrane-penetrating, and/or autophilic properties. The conjugation of certain peptides to antibodies has been shown to increase the potency of antibodies, e.g., in inducing apoptosis (Zhao, et al. 2001; Zhao, et al 2002a; Zhao, et al. 2002b). The conjugation chemistry used in previous studies has utilized the nucleotide binding site (Pavlinkova, et al. 1997) or the carbohydrate moiety of antibodies as the site of specific attachment (Award, et al. 1994).

In efforts to enhance antigen detection and/or therapeutic efficacy of known antibodies, many hybrid molecules comprising two distinct covalently linked domains have been proposed. For instance, U.S. Pat. No. 5,219,996 (issued to Bodmer et al.) proposes changing an amino acid residue of an antibody molecule to a cysteine residue and then coupling an effector or reporter molecule to the antibody through the cysteine thiol group. U.S. Pat. No. 5,191,066 (issued to Bieniarz et al.) proposes periodate oxidation of a carbohydrate molecule in the Fc region of an immunoglobulin and attaching a disulfide compound thereto. U.S. Pat. No. 6,218,160 (issued to Duan) proposes site-specific conjugation of an enzyme to an antibody by formation of a dihydrazone bridge therebetween. U.S. Pat. No. 5,596,081 (issued to Haley et al.) discloses a method for site-specific attachment of a purine or purine analog photoaffinity compound to an antibody molecule. U.S. Pat. No. 6,238,667 (issued to Kohler) proposes photochemically cross-linking an azido-peptide molecule to an antibody at a purine or tryptophan affinity site on the antibody. U.S. Patent Pub. No. 2005/0033033 (Kohler et al.) proposes a super-antibody for inhibiting cell apoptosis, wherein the super-antibody comprises an anti-caspase antibody conjugated to a membrane transporter peptide. U.S. Patent Pub. No. 2003/0103984 (Kohler) discloses a fusion protein comprising antibody and peptide domains in which the peptide domain can have autophilic activity. U.S. Pat. No. 6,482,586 (issued to Arab et al.) proposes covalent hybrid compositions for use in intracellular targeting. U.S. Pat. No. 6,406,693 (issued to Thorpe et al.) proposes antibodies and conjugates for cancer treatment by binding to aminophospholipid on the luminal surface of tumor blood vessels. U.S. Pat. No. 6,780,605 (issued to Frostegard) proposes a method of diagnosing cardiovascular disease that employs antibodies specific for platelet activating factor. U.S. Pat. No. 6,716,410 (issued to Witztum et al.) proposes a treatment for atherosclerosis that employs a monoclonal antibody having specific binding affinity for oxidized low density lipoprotein (oxLDL), which is covalently linked to a therapeutic agent, e.g., a thrombolytic agent. U.S. Patent Pub. No. 2003/0143226 (Kobayashi et al.) proposes a monoclonal antibody having specific binding affinity for an oxidized LDL receptor, which inhibits binding of oxLDL to the receptor.

The above approaches are proposed to enhance the antigen detection ability and/or therapeutic efficacy of antibodies, which are not sufficiently effective in locating or killing their targets in either their native or “humanized” states. Still, there continues to be a need for enhancing the detection, prevention and/or treatment of many diseases using suitably modified antibodies. An object of the present invention is to address the foregoing needs with suitably prepared super-antibodies.

### SUMMARY OF THE INVENTION

The present invention affords novel super-antibodies having autophilic, membrane-penetrating, adjuvant, and/or catalytic properties. A super-antibody contemplated by the present invention comprises immunoglobulin (Ig) and non-immunoglobulin (non-Ig) domains, wherein at least one non-Ig domain is covalently attached to the Ig domain, preferably as a chemically formed hybrid molecule, i.e., an immunoconjugate. The immunoglobulin domain can comprise a polyclonal antibody, monoclonal antibody, Fab fragment, or F(ab′)₂ fragment, which imparts specific binding affinity for an antigenic determinant. The non-Ig domain is an organic chemical moiety that imparts, or augments, autophilic, membrane-penetrating, adjuvant, and/or catalytic properties to the immunoconjugate, but which does not contain an azido, purine or pyrimidine group. Preferably, the non-Ig domain comprises a peptide having autophilic, membrane-penetrating, adjuvant, and/or catalytic properties.

Another aspect of the present invention is directed to a method of making novel super-antibodies. In a method of the invention, a photoactivatable organic molecule is covalently linked to an immunoglobulin at a site on the immunoglobulin having binding affinity for the organic molecule. The mutual attraction of 1g and photoactivatable organic molecule favors contact and coupling of the two entities upon exposure to activating radiation. Preferably, the organic molecule contains a chromophore, such as an aromatic hydrocarbon moiety, other than a purine or pyrimidine group, susceptible to photoactivation. Also, an azido group need not be present in the molecule.

Preferably, an aromatic hydrocarbon moiety (AHM) of the invention, which is photoactivatable, is a single ring or polynuclear aryl or heterocycle. Inclusive of such moieties are substituted benzene, naphthalene, anthracene, phenanthrene, pyrrole, furan, thiophene, imidazole, pyrazole, oxazole, thiazole, pyridine, indole, benzofuran, thionaphthene, quinoline, or isoquinoline groups. Conveniently, an AHM is present in the photoactivatable organic molecule as part of a side chain of an amino acid residue. Exemplary of such amino acid residues are tryptophan, tyrosine, histidine, and phenylalanine, which have indole, phenol, imidazole, and phenyl side chains, respectively. A tryptophan residue is most preferred.

A super-antibody of the invention can also be conjugated with one or more non-autophilic peptides to add functionality. For instance, a super-antibody can bear a membrane-penetrating peptide sequence, which facilitates translocation of the antibody across the cell membrane where it can bind to an intracellular target. In a specific embodiment, the membrane-penetrating peptide comprises at least one MTS peptide or MTS-optimized peptide. Additionally, an autophilic super-antibody can be conjugated with a membrane-penetrating peptide sequence, thereby imparting both functionalities to the antibody.

In another aspect of the present invention, a super-antibody having specific binding affinity for atherosclerotic plaques, which permits detection, prevention and/or treatment of atherosclerosis, is contemplated. For example, an autophilic super-antibody is capable of binding an antigenic determinant of atherosclerotic plaques, e.g., ox-LDL, and can dimerize or oligomerize once specifically bound to its antigenic determinant. In this way, uptake of ox-LDL by macrophages can be effectively blocked or reduced, thereby inhibiting chronic inflammation associated with atherosclerosis. In specific embodiments, an autophilic peptide of the immunoconjugate comprises a T15, T15-scr2, R24, R24-charged, or other optimized amino acid sequence. Preferably, the immunoglobulin and/or peptide domains of the super-antibody are humanized to improve tolerance in a patient.

A pharmaceutical composition is also contemplated, which contains one or more super-antibodies and a pharmaceutically acceptable carrier. Due to its superior avidity, a super-antibody of the invention can be administered to a patient in a dosage similar to, or less than, that practicable for the corresponding non-autophilic antibody.

In another aspect of the invention, an assay of cells undergoing apoptosis can be performed by contacting the cells with a super-antibody of the invention. The super-antibody specifically binds to an antigenic determinant of a cell undergoing apoptosis and can be visualized by a reporter molecule or secondary antibody. Exemplary of antigenic determinants associated with apoptosis are membrane-bound phosphorylcholine and phosphatidylserine.

### DESCRIPTION OF DRAWINGS

FIG. 1 compares the internalization of MTS conjugated antibodies and non-MTS conjugated antibodies using anti-caspase 3 antibodies.

FIG. 2 depicts the effect of chemotherapeutic drug (actinomycin D) on cell death in the presence and absence of MTS-conjugated (Sab) antibody.

FIG. 3 depicts enhanced binding of anti-CD20 antibodies conjugated with T15 peptide.

FIG. 4 depicts improved binding of anti-CD20 antibodies conjugated with T15 peptide at low concentrations of antibody.

FIG. 5 depicts improved binding of anti-CD20 antibodies conjugated with T15 peptide to DHL-4 cells at high concentrations of antibody.

FIG. 6 depicts enhanced induction of apoptosis of tumor cells with mouse anti-CD20 conjugated with T15 peptide.

FIG. 7 compares the binding of anti-GM2 antibody and T15 conjugated anti-GM2 antibody to ganglioside GM2.

FIG. 8 illustrates the self-binding activity of anti-GM2 antibody and T15 conjugated anti-GM2 antibody.

FIG. 9 demonstrates binding specificity of T15 conjugated anti-GM2 antibody to different gangliosides.

FIG. 10 depicts differences in cell surface binding of anti-GM2 antibody and T15 conjugated anti-GM2 antibody to Jurkat cells.

FIG. 11 depicts the effect of anti-GM2 antibody and T15 conjugated anti-GM2 antibody on Jurkat cell growth.

FIG. 12 compares the efficacy of autophilic peptide conjugation to an affinity site on an antibody (nucleotide) vs. a non-affinity site (CHO-carbohydrate) using anti-GM2.

FIG. 13 depicts enhanced apoptosis of tumor cells using anti-GM2 antibody conjugated with T15 peptide.

FIG. 14 compares the binding of Herceptin® (upper panel) and the autophilic peptide conjugated form of Herceptin (lower panel) to small cell lung cancer cell.

FIG. 15 depicts photo-conjugation of biotin-amino acids to monoclonal OKT3 antibody. A panel of biotin-amino acids were mixed with the monoclonal antibody OKT3 at concentration from 20-50 μMol and exposed to UV for 2 minutes. The reacted mixture was dot-blotted with avidin-HRP and scanned. Color intensity is indicated at the y-axis.

FIG. 16. Panel A: Titration of biotin-tryptophan photo-conjugation to chimeric anti-GM2 antibody. Chimeric anti-GM2 was photo-biotinylated with Trp peptide at different molarities. ELISA wells were incubated with chimeric biotinylated anti-GM2 blocked and developed with avidin-HRP. Panel B: Photobiotinylation of humanized anti-Her2/neu (Herceptin) with Trp-biotin peptide under different pH, ELISA as in Panel A.

FIG. 17. Denaturation of photo-biotinylated anti-GM2 antibody. Detection of biotin on denatured/renatured antibody in ELISA as in FIG. 16A.

FIG. 18. Panel A: Comparison of single versus multiple biotin anti-GM3 antibody. ELISA wells were coated with ganglioside, single and multiple biotin anti-GM3 was added and developed with avidin-HRP. Panel B: Comparison of single versus multiple biotin chimeric anti-Gm2 antibody to Gm2. Comparison of single versus multiple biotin antibody. ELISA as in FIG. 19.

FIG. 19 compares chemically biotinylated with photo-biotinylated antibodies. Commercial NHS-biotin rabbit anti-mouse (Sigma) and NHS-biotin anti-GM2 are compared with photobiotinylated antibodies. ELISA as in FIG. 16.

FIG. 20 compares detection sensitivity of photo- and chemically biotinylated chimeric anti-glycolyl GM3 binding to glycolyl GM3 monoganglioside. ELISA as in FIG. 19.

FIG. 21 demonstrates antigen specific binding of photobiotinylated anti-glycolyl GM3 to monogangliosides GM1, GM2, GM3 and glycolyl GM3. ELISA as in FIG. 20.

FIG. 22 illustrates a proposed mechanism by which an autophilic antibody of the present invention, which is immunospecific for ox-LDL, can inhibit chronic inflammation leading to atherosclerosis.

### DESCRIPTION OF THE INVENTION

### SuperAntibody Synthesis and Formulations

It has now been discovered that many immunoglobulins have an affinity for certain photoactivatable aromatic hydrocarbon moieties. Such affinity permits close approach and prolonged contact time between the immunoglobulin (Ig) and the aromatic hydrocarbon moiety (AHM), which in turn facilitates photolytic conjugation of the Ig to an organic molecule bearing the AHM. Without wishing to be bound to any particular theory, it is believed that the attraction between the AHM and an affinity site on the Ig is probably due to van der Waals forces and/or dipole-dipole interactions, which promote the close approach and stacking of parallel aromatic rings.

In the present invention, a photoactivatable organic compound is covalently linked to an Ig to form an immunoconjugate (super-antibody). Such immunoconjugate is formed by admixing the photoactivatable organic compound and Ig, and subjecting the admixture to photoactivation conditions effective to covalently link the photoactivatable organic compound to the Ig. A photoactivatable organic compound of the present invention contains at least one AHM, which has a binding affinity for the Ig. However, the photoactivatable organic compound does not contain an azido, purine or pyrimidine group, inasmuch as such groups may interact with a different affinity site on the Ig, or may unnecessarily complicate synthesis of the photoactivatable organic compound.

In a preferred aspect of the invention, in addition to an AHM, a photoactivable organic compound comprises a peptide having self-binding, membrane-penetrating, adjuvant, and/or enzymatic properties. Such peptide can thereby impart its properties to a subsequently formed immunoconjugate. Preferably, a photoactivable organic compound comprising a peptide contains from about 5 to about 30 amino acid residues.

In a further preferred aspect of the invention, a peptide contains an autophilic amino acid sequence selected from the following group:

(SEQ ID NO: 1)

NH-ASRNKANDYTTDYSASVKGRFIVSR-COOH,

(SEQ ID NO. 4)

NH-SKAVSRFNAKGIRYSETNVDTYAS-COOH,

(SEQ ID NO. 5)

NH-GAAVAYISSGGSSINYA-COOH,
and

(SEQ ID NO. 6)

NH-GKAVAYISSGGSSINYAE-COOH.

Alternatively, a peptide contains a membrane-penetrating amino acid sequence selected from the following group:

(SEQ ID NO. 2)

NH-KGEGAAVLLPVLLAAPG-COOH,
and

(SEQ ID NO. 7)

NH-WKGESAAVILPVLIASPG-COOH.

An AHM covalently linked to a peptide in a photoactivatable organic compound is preferably located at a C- or N-terminus of the peptide so as not to interfere with the desired properties of the peptide. Conveniently, the AHM can be present in an aromatic side chain of an amino acid, such as tryptophan, tyrosine, histidine, and phenylalanine.

As referred to herein, an “immunoglobulin” can be a polyclonal antibody, monoclonal antibody, Fab fragment, or F(ab′)₂ fragment. It is generally preferred that mutual attraction and covalent linkage between the Ig and AHM occurs at an affinity site located in a variable domain of the immunoglobulin. For autophilic peptides, this can ensure close approach and noncovalent interaction between two adjacent Ig molecules on a cell surface. Such coupling of 1g molecules can, in turn, facilitate crosslinking of cellular receptors and promote intracellular signaling. Similarly, for membrane-penetrating peptides, location of the peptide adjacent a cellular receptor for the peptide can facilitate transport of an immunoconjugate into the cell. Binding affinity between the Ig and AHM can be demonstrated, as shown hereinafter, by competitive binding with an aromatic reporter molecule also having affinity for the Ig binding site. In practice, due to a multiplicity of affinity sites on the immunoglobulin, a plurality of photoactivatable organic compounds can be covalently linked to the Ig. Functionally, any type of immunoglobulin can be employed with the present invention, such as those having specific binding affinity for a cancer-related antigen, a caspase enzyme, ox-LDL, or cellular receptor.

An aromatic hydrocarbon moiety (AHM) of the present invention comprises at least one aryl, polynuclear aryl, heterocycle, or polynuclear heterocycle group. Representative of these different chemical classes are the following functional groups: aryl-benzene; polynuclear aryl-naphthalene, anthracene, and phenanthrene; heterocycle-pyrrole, furan, thiophene, pyrazole, oxazole, thiazole, pyridine, and imidazole,; polynuclear heterocycle-benzofuran, acridine, thionaphthene, indole, quinoline, and isoquinoline, and geometric isomers thereof. Thus, for embodiments in which a photoactivatable organic compound comprises a peptide covalently bonded to an AHM, the AHM can be present in an amino acid residue of the peptide, e.g., tryptophan (indole), tyrosine (substituted benzene), histidine (imidazole), and phenylalanine (benzene). Representative AHMs are illustrated in Table 1.

Also contemplated within the invention is a pharmaceutical composition that comprises a pharmacologically effective amount of an instant super-antibody and a pharmaceutically acceptable carrier. Representative of such carriers are saline solution, e.g., 0.15% saline solution.

In a preferred embodiment, a photoreactive biotinylated tryptophan is inserted into several antibodies to yield biotinylated antibodies. This biotinylation reaction is not inhibited by the presence of ATP, which is a ligand for the conserved nucleotide binding site on antibodies (Rajagopalan, et al., 1996), and suggests that a different affinity site is involved. Moreover, it has been reported that UV energy can induce reactive radicals in heterocyclic compounds, such as tryptophan (Miles, et al. 1985). Thus, in a preferred embodiment of the present invention, UV light is used to covalently attach tryptophan-containing molecules to antibodies at a tryptophan affinity site on the antibodies.

TABLE 1
Aromatic Hydrocarbon Moieties.

| | |
|---|---|
| Benzene | 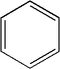 |
| Anthracene | 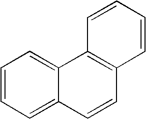 |
| Phenanthrene | 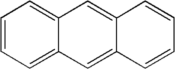 |
| Acridine | 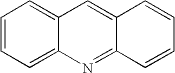 |
| Pyrazole | 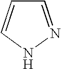 |
| Thiazole | 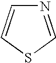 |
| Imidazole | 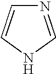 |
| Thionaphthene | 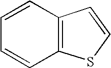 |
| Indole | 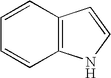 |
| Naphthalene | 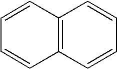 |
| Pyrrole | 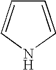 |
| Furan | 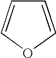 |
| Thiophene | 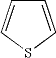 |
| Oxazole | 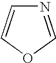 |
| Pyridine | 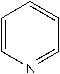 |
| Benzofuran | 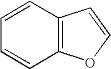 |
| Quinoline | 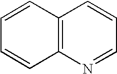 |
| Isoquinoline | 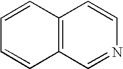 |

With the discovery of an affinity of antibodies for AHMs, such as tryptophan, a simple, gentle and rapid method is available to conjugate organic molecules to antibodies. A practical application is the use of multiple biotinylated AMHs to affinity biotinylate antibodies. Additionally, AHM-containing peptides having biological or chemical properties can be conveniently affinity cross-linked to antibodies to create super-antibodies.

Alternative methods of synthesizing antibody conjugates employ chemical or genetic engineering techniques to couple a peptide to an antibody. For instance, a peptide can be attached by chemical means to an immunoglobulin (whole polyclonal or monoclonal antibody, or fragment thereof) at a carbohydrate site of the Fc portion or to an amino or sulfhydryl group of an antibody. Additionally, a peptide can be coupled to an antibody's variable domain structures by photo-crosslinking an azido-tryptophan or azido-purine to the antibody. In the latter approach, the peptide is believed to attach preferentially to the antibody by photoactivation of the azido group at a tryptophan or purine affinity site. In a further approach, a chimeric antibody can be expressed, using genetic manipulation techniques, as a fusion protein of an autophilic peptide and a whole immunoglobulin, or fragment thereof. See, e.g., U.S. Pat. No. 6,238,667, PCT Publ. WO 9914244, U.S. Pat. RE 38,008, U.S. Pat. No. 5,635,180, and U.S. Pat. No. 5,106,951, the disclosures of which are incorporated herein by reference.

Autophilic antibodies of the present invention typically comprise antibodies conjugated with one or more peptides having an autophilic sequence. It is believed that an autophilic antibody of the invention can comprise virtually any immunoglobulin. In some embodiments, the antibodies bind to targets implicated in a disease or disorder, where binding of the target has a therapeutic effect on the disease or disorder. The target antigens can include cell-surface antigens, including trans-membrane receptors. In specific embodiments, the Ig component of the antibodies can comprise the monoclonal antibody 5D10 which binds human B-cell receptors, the monoclonal antibody S1C5 which binds murine B-cell receptors, anti-CD20 antibodies such as rituximab (Rituxan®) which binds CD20 on normal and malignant pre-B and mature B lymphocytes, mouse monoclonal antibody IF5 which is specific for CD-20 on human B-cell lymphomas, tositumab (Bexxar®) which also binds CD20 on B lymphocytes, anti-GM2 which binds human ganglioside GM2 lymphocytes, trastuzumab (Herceptin®) which binds the protein HER2 that is produced by breast cells, anti-caspase antibodies which recognize the caspase proteins involved in apoptosis, humanized TEPC-15 antibodies which are capable of binding oxidized low density lipoproteins (ox-LDL) and can prevent uptake of oxidized LDL by macrophages, humanized T15-idiotype positive antibodies which bind phosphocholine, and humanized R24 antibodies which recognize the human GD3 ganglioside on melanoma cell surfaces.

An autophilic antibody of the present invention can comprise any autophilic peptide sequence. The autophilic peptide can also comprise optimized peptide sequences, which may include sequences having enhanced functionality, such as those that act as linkers to enhance display and cross-linking activity of antibodies, or residues that enhance solubility of autophilic sequences.

The present invention contemplates a method of producing an autophilic conjugate of the invention in which a template peptide has been modified to enhance the crosslinking potential of the autophilic antibodies as described above. In one embodiment of the invention, such functionally enhanced peptides are determined by producing a series of synthetic peptides with substitutions at each amino acid position within the template sequence and then testing this library of peptides for autophilic binding or for binding to the original peptide sequence. Those peptides with superior binding to the original sequence are then conjugated to immunoglobulins and the resultant conjugates are tested for potency, specificity, and the unwanted ability to induce aggregation. In one specific embodiment, the T15 peptide sequence is altered and modified sequences are selected for enhanced function.

In another embodiment of the invention, the self-binding potential of a peptide can be enhanced by increasing complementarity of the sequence, such as described in U.S. Pat. No. 4,863,857 (issued to Blalock et al.), which is incorporated herein by reference. The self-binding potential and/or toleration of a peptide can also be enhanced by humanizing a self-binding peptide sequence derived from non-human animals. Humanizing a peptide sequence involves optimizing the sequence for expression or functionality in humans. Examples and methods of humanizing peptides and proteins have been described elsewhere (Roque-Navarro et al., 2003; Caldas et al., 2003; Leger et al., 1997; Isaacs and Waldmann, 1994; Miles et al. 1989; Veeraraghavan et al., 2004; Dean et al., 2004; Hakenberg et al., 2003; Gonzales et al., 2004; and H. Schellekens, 2002).

In a preferred embodiment, an autophilic peptide comprises the T15 peptide, which originally comprised regions of CDR2 and FR3 of the murine germline-encoded S107/TEPC15 antibody. The T15 peptide comprises the amino acid sequence: ASRNKANDYTTDYSASVKGRFIVSR (SEQ ID NO.: 1) (Kang C-Y, et al., 1988). Its autophilic property has been shown to be antigen-independent, thereby suggesting attachment of the peptide to monomeric antibodies can impart autophilic and increased avidity properties to the antibodies (Kaveri S., et al., 1991). The T15 peptide can be photo-crosslinked to an aromatic hydrocarbon moiety or nucleotide affinity site of the immunoglobulin to produce the autophilic antibody. Alternatively, the T15 peptide can be crosslinked to a carbohydrate site of the Fc portion or to an amino or sulfhydryl group of the immunoglobulin. Also, the autophilic antibody can be conveniently expressed as a fusion protein of the T15 peptide and whole immunoglobulin, or fragment thereof. In other specific embodiments, an autophilic peptide can comprise the scrambled T15 sequence (T15-scr2), which comprises the amino acid sequence NH-SKAVSRFNAKGIRYSETNVDTYAS-COOH (SEQ ID NO. 4), the peptide R24 comprising the sequence NH-GAAVAYISSGGSSINYA-COOH (SEQ ID NO. 5), the peptide R24-charged comprising the sequence NH-GKAVAYISSGGSSINYAE-COOH (SEQ ID NO. 6), and any modifications to such peptides which optimize or enhance the binding and therapeutic effectiveness of antibodies.

The attachment of autophilic peptide to a monomeric antibody can impart autophilic and increased avidity properties to the antibody (Y. Zhao, and H. Kohler, 2002). In specific embodiments, the antibody can be a humanized version of an orthologous antibody, which acquires increased or optimized binding and effectiveness when conjugated to an autophilic peptide, such as one containing the T15 sequence. Methods of humanizing antibodies have been previously described. See, e.g., U.S. Pat. No. 5,639,641 (issued to Pedersen et al.), U.S. Pat. No. 5,498,531 (issued to Jarrell), U.S. Pat. Nos. 6,180,370 and 5,693,762 (issued to Queen et al.), which are incorporated herein by reference.

Autophilic antibody conjugates of the present invention can also comprise one or more other bioactive or functional peptides, which confer additional functionality on the antibody conjugates. For example, the antibody conjugate can comprise an antibody that bears a T15 autophilic peptide and an MTS membrane translocation peptide (Y. Zhao et al., 2003; Y. Lin et al., 1995). In a specific embodiment, the MTS translocation peptide can have the amino acid sequence KGEGAAVLLPVLLAAPG (SEQ ID NO. 2). In another embodiment, the translocation peptide can be an optimized MTS peptide, comprising the amino acid sequence WKGESAAVILPVLIASPG (SEQ ID NO. 7). The T15 peptide provides autophilicity to the conjugate, and the MTS sequence facilitates entry of the antibody into cells. Such a conjugate can target, for example, cancer cells for radio-immunotherapy, when its antibody region targets a primarily intracellular, tumor-associated antigen, such as carcino-embryonic antigen (CEA). See, e.g., U.S. Pat. No. 6,238,667, which is incorporated herein by reference. The autophilic conjugate, upon administration, targets CEA-bearing, colon carcinoma cells, is internalized by translocation of the antibody mediated by the MTS peptide, and is enabled to bind to the more prevalent intracellular form of CEA. Crosslinking of CEA antibody with, for instance, a therapeutic isotope such as ¹³¹I can be retained in a cell longer than unmodified, labeled antibody and can deliver a higher radioactive dose to the tumor. In addition, such therapeutic isotopes as ¹²⁵I, which release beta particles of short path length and are not normally considered useful for therapy, can, when delivered intracellularly in closer proximity to the nucleus, be efficacious against certain targets, especially those of lymphoid origin and accessible in the blood and lymph tissues. Other categories of secondary, bioactive or functional peptides include peptides capable of binding to receptors, and peptide mimetics, capable of binding to a distinctive antigen or epitope of the same antigen, targeted by the primary antigen combining site.

Autophilic antibodies conjugated with one or more other functional peptides may also be useful for targeting intracellular antigens. Such antigens could include tumor associated antigens and viral proteins. For example, an autophilic antibody specific for viral proteins which is conjugated with a self-binding peptide and a MTS peptide can also be used to bind to intracellular viral proteins and prevent production of viruses. The antibody can be internalized through the MTS peptide, and can be optimized to bind intracellular viral proteins (Zhao, Y., et al. 2003). Many other functional peptides may also be conjugated to the autophilic antibodies to increase functionality.

The invention also relates to compositions comprising a super-antibody of the invention and a pharmaceutically acceptable carrier. Conjugate autophilic antibodies can bind non-covalently with other autophilic antibodies when bound to their target antigen(s). However, premature formation of dimers or multimers of the antibodies may lead to difficulties in manufacturing, such as during purification and concentration, as well as drawbacks in administration, which may lead to side effects. As such, compositions containing autophilic antibody-peptide conjugates of the invention are formulated to reduce this dimerizing potential and maximize monomeric properties while in solution and before administration. For example, it has been found that solution dimerization can be reduced or mitigated by using a hypertonic composition. In some embodiments, salt concentrations of 0.5M or more, low levels of SDS or other various detergents such as those of an anionic nature (see U.S. Pat. No. 5,151,266, which is incorporated herein by reference), or modifications of the antibody to decrease its isoelectric point, for example through the use of succinyl anhydride (see U.S. Pat. No. 5,322,678, which is incorporated herein by reference), can be used to formulate compositions.

### Disease Detection, Prevention and Treatment

A method of enhancing apoptosis, complement fixation, effector cell-mediated killing of targets, or preventing the development of, or enhancement of, a disease state, is also contemplated, which employs a super-antibody of the invention or a composition comprising the super-antibody. In one embodiment, an autophilic conjugate of the invention, or a composition containing an autophilic conjugate of the invention, is administered to a subject. Once administered, the antibodies bind to target cells and enhance apoptosis, complement fixation, effector cell-mediated killing of targets, or prevent target antigens or cells from stimulating the development of, or further enhancing, a disease state. In a further embodiment, allowing time for the autophilic conjugate to bind to target cells and enhance apoptosis, complement fixation, effector cell-mediated killing of targets, or prevent target antigens or cells from further enhancing a disease state, and for the autophilic conjugate to be cleared from normal tissues, a second anti-autophilic peptide antibody can be administered. For example, if an autophilic conjugate contains a non-native autophilic peptide, such as the murine T15 sequence, an anti-T15 peptide antibody can be administered, which recognizes and binds to antibodies conjugated with the T15 sequence. This allows binding to and enhancement of apoptosis of pre-localized super-antibodies. Additionally, a template autophilic peptide can be modified to enhance the crosslinking potential of the autophilic antibodies as described above.

In another aspect of the invention, a method of potentiating apoptosis of targeted cells of a patient comprises administering a first autophilic antibody-peptide conjugate, or a composition containing an autophilic antibody-peptide conjugate, and a second antibody, or composition containing the second antibody, which recognizes the autophilic peptide domain of the conjugate. In this embodiment, the antibody-peptide conjugate recognizes an antigen on a target cell. Owing to its homodimerization property, the antibody-peptide conjugate can bind more avidly to the target than the corresponding antibody lacking the autophilic peptide domain. This is likely due to the ability to crosslink antigen at the surface of target cells. Moreover, whenever the autophilic antibodies bind to two or more antigens, with those antigens being brought in close proximity and crosslinked, due to the autophilic property of the antibodies, an apoptosis signal within the cell can be triggered. In those instances when the peptide domain of the conjugate presents an exposed epitope, a second antibody, specific for the autophilic peptide, can be administered, bind to the modified antibody, and enhance the process of crosslinking and even cause temporary clearance of the target antigen. As an example, if the target antigen is a receptor, clearance from the cell surface, endocytosis, and degradation will subsequently require synthesis of new receptor protein, meaning that the biological function of the receptor will be more effectively inhibited for a longer period than using either a simple blocking antibody or small molecule inhibitor. Alternatively, the second antibody can bear a radiolabel or other potentially therapeutic substance, so that when administered, it can attack the targeted cells. Since the autophilic peptide is present on only a small number of immunoglobulins and may be derived from another organism, the secondary antibody should have specificity for antibodies bearing the autophilic peptide. Thus, antibody specific to the autophilic peptide will have the requisite selectivity to be used in vivo.

In another aspect of the invention, a patient who suffers from a disease or condition responsive to antibody therapy is administered at least one autophilic antibody of the invention in an amount effective to alleviate symptoms of the disease or condition. A disease or condition contemplated for treatment by an antibody of the invention can be a malignancy, neoplasm, cancer, atherosclerosis, auto-immune disorder, Alzheimer's disease or other neuro-degenerative condition, graft or transplantation rejection, or any other disease or condition responsive to antibody therapy.

Atherosclerosis is a major cause of fatal and chronic vascular diseases that include stroke, heart failure and disruption of circulation in other organs and sites. There is increasing evidence that atherosclerosis is a chronic inflammatory disease. Recent findings indicate that oxidized lipids, especially phospholipids but also oxysterols, generated during LDL oxidation or within oxidatively stressed cells, are triggers for many of the events seen in developing lesions (Libby, P., et al., 2003). Oxidized phospholipids in ox-LDL are ligands for scavenger receptors on macrophages (Horkko, S., et al., 2000). Thus, ox-LDL and its products, including but not limited to the oxidized phospholipids and oxysterols, are initiating factors to which the artery wall and its component cells respond. The classical lipid hypothesis and the new inflammation hypothesis should be jointly considered part of the pathogenetic pathway in atherosclerosis.

One aspect of the present invention aims to block the inflammatory pathway, thereby halting further plaque formation in patients with high cholesterol and lipid levels. In a preferred embodiment, a mouse T15 antibody is “humanized” into a therapeutic antibody to treat vascular diseases in humans. Humanization of non-human antibodies may require extensive re-shaping of the antibody molecule, which can result in loss or reduction of antibody specificity and affinity. By conjugating an autophilic peptide to a humanized T15 antibody, its superb targeting for ox-LDL can be restored, thereby blocking uptake of ox-LDL by macrophages and inhibiting chronic inflammation associated with atherosclerosis. A humanized T15 specific for ox-LDL thereby mimics the human body's autoantibody response to the same antigen, which may be diminished in immune-compromised individuals.

Accordingly, a general method of preventing or treating atherosclerosis in a patient comprises administering to the patient a super-antibody having specific binding affinity for oxidized low density lipoprotein (ox-LDL) and autophilic properties. The super-antibody is administered at a dose effective to block or reduce uptake of ox-LDL by macrophages, thereby inhibiting chronic inflammation associated with atherosclerosis. Preferably, the immunoconjugate specifically binds phosphorylcholine and expresses the T15 idiotype. The immunoconjugate can be humanized, and preferably contains an autophilic peptide sequence, such as SEQ ID NO: 1, SEQ ID NO: 4, SEQ ID NO: 5, and SEQ ID NO: 6.

According to the principles of the present invention, a super-antibody, or a composition containing a super-antibody, is preferably administered in one or more dosage amounts substantially identical to, or lower than, those practicable for unmodified antibodies. Thus, in the treatment of a lymphoma or a breast cancer, an autophilic antibody of the invention can be administered in one or more dose amounts substantially identical to, or less than, the doses used for rituximab or trastuzumab. For example, treatment with trastuzumab (a humanized monoclonal anti-HER2/neu antibody) in a patient with HER2⁺ breast cancer employs an antibody concentration of about 10 mg/ml. Intravenous infusion over 90 minutes provides a total initial dose of 250 mg on day 0. Beginning at day 7, 100 mg is administered weekly for a total of 10 doses. The dosing regimen is reduced gradually from 250 mg to 100 mg to a maintenance dose of 50 mg per week. Similar or lower dosage regimens to that for trastuzumab can be employed with autophilic antibodies, with any adjustments being well within the capabilities of a skilled practitioner.

In a preferred embodiment, a super-antibody of the present invention has a specific binding affinity for oxLDL. Exemplary of an antibody domain of the super-antibody is the monoclonal antibody 1K17, as described by U.S. Pat. No. 6,716,410 (issued to Witztum et al.), the pertinent disclosure of which is incorporated herein by reference. When modified with an autophilic peptide according to the principles of the present invention, the resulting superior avidity of the autophilic antibody can enhance the binding property of the antibody absent the peptide. An autophilic antibody can localize to oxLDL of atherosclerotic plaques, whereupon it can be employed to detect the situs of the plaque when used with a label, reporter molecule, or secondary antibody, and the like. Alternatively, an autophilic antibody can be employed to coat the site of oxLDL deposition, thereby preventing further accumulation of plaque. In yet another aspect, an autophilic antibody can be employed to direct an anti-plaque agent, e.g., a thrombolytic or antioxidant agent.

Witztuin et al. have reported that a human antibody fragment (Fab), referred to as IK17, binds to an epitope of ox-LDL and a breakdown product, MDA-LDL, but not native LDL. Moreover, they propose the Fab can inhibit uptake of ox-LDL by macrophages, presumably by binding to an epitope on ox-LDL that is recognized by macrophage scavenger receptors. The Fab is therefore proposed to inhibit atherogenesis by blocking the inflammatory response. These authors also report that anti-ox-LDL human antibodies express the so-called T15 idiotype (Shaw, P., et al., 2000). The T15 idiotype was originally described as being specific for phosphorylcholine (Lieberman, et al., 1974). Previously, it was discovered that the T15 idiotype is autophilic, i.e., they self-associate as noncovalent dimers (Kaveri, S., et al., 2000). By coupling the autophilic T15 peptide to a humanized T15/S107 antibody, the self-binding properties of the T15 antibody and its avidity can be restored.

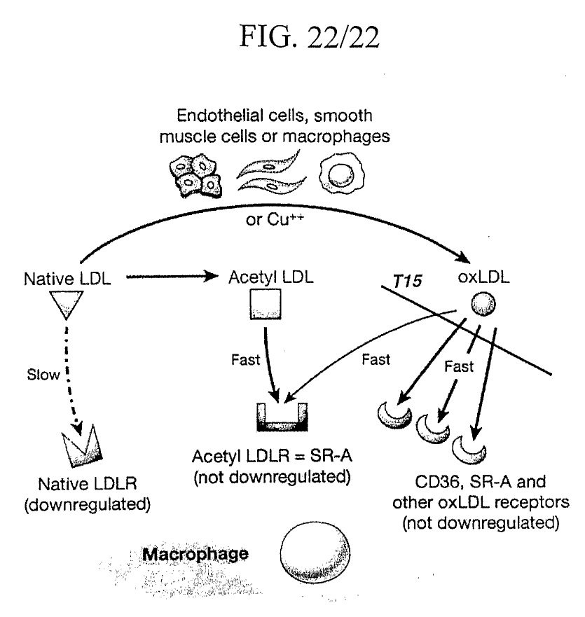

Upon showing that the T15 antibody is biologically equivalent to the human anti-phosphorylcholine antibodies known to bind to ox-LDL and inhibit inflammation initiated by macrophages, the efficacy of the T15 antibody in preventing and/or treating atherosclerosis is demonstrated. A proposed mode of action of the T15 antibody is schematically indicated in FIG. 22 (modified from Steinberg, Nature Medicine, 2002, 8: 12311).

The present invention is also for a method of detecting a disease state, such as the presence of atherosclerotic plaques in a patient's vascular system. Such method comprises administering to a patient an immunoconjugate of the present invention, which has a specific binding affinity for oxidized low density lipoprotein (ox-LDL). The immunoconjugate also has autophilic properties. Sites of immunoconjugate concentration in the patient's vascular system are then detected, thereby localizing and visualizing the atherosclerotic plaques. Preferably, the immunoconjugate binds phosphorylcholine and/or expresses the T15 idiotype. More preferably, the immunoconjugate bears an autophilic peptide having an aforementioned amino acid sequence.

A method of detecting cells undergoing apoptosis, which may be indicative of a disease state, is also contemplated. For example, when an antigenic determinant of a cell surface is represented by membrane-bound phosphorylcholine or phosphatidylserine, the cell can be contacted with an autophilic immunoconjugate of the invention, which has specific binding affinity for the antigenic determinant. The presence or absence of immunoconjugate bound to the cell is then detected. Previously described autophilic peptides can be used. Such methods as flow cytometry, fluorescent microscopy, histological staining, or in vivo imaging are particularly preferred for conducting detection. To facilitate these, the immunoconjugate may be labeled with fluorescein.

Additionally, an in vitro assay of apoptosis can be used to screen multiple antigen-positive target cell lines, and if possible, fresh isolates of antigen-positive cells. A non-modified antibody is incubated with a secondary (anti-immunoglobulin) antibody to enhance the potential for cross-linking. Cells may be enumerated by pre-labeling, such as with ⁵¹Cr or ¹³¹I-UDR, or by FACS analysis using indicators of apoptosis. Positive results in this assay predict a positive outcome using an autophilic immunoconjugate. However, negative results in the assay do not necessarily mean that subsequent conjugation with an autophilic peptide will not improve one or more antibody effector properties.

Autophilic antibodies of the present invention have a higher potential for forming dimers in vitro under laboratory conditions, such as in solution with PEG. This laboratory characteristic correlates with crosslinking ability upon binding to a cell-surface target and higher therapeutic potency through such mechanisms as triggering apoptosis. This characteristic can be used to identify natural SuperAntibodies and to screen for proper conjugation of self-binding peptides to a non-autophilic antibody. Suitable animal models for testing efficacy of the aforementioned autophilic antibodies include severely compromised immunodeficient (SCID) mice or nude mice bearing human tumor xenografts.

The following examples are presented to illustrate certain aspects of the invention, and are not intended to limit the scope of the invention.

### EXAMPLES

### Example 1

### Conjugation of T15 Peptide to Two Mabs Specific for B-Cell Receptor

Cell Line and Antibodies.

The human B-cell tumor line (Su-DHL4) and murine B-cell tumor line (38C13) are grown in RPMI 1640 medium (supplemented with 10% fetal bovine serum, 2 μmol/L glutamine, 10 μmol/L HEPES, 50 U/mL penicillin, and 50 μg/mL streptomycin, 50 μmol/L 2-mercaptoethanol) at 37° C. under 5% carbon dioxide. Two mAbs, 5D10 and S1C5, specific for the human or murine BCR, respectively, were used in this study. The antibodies are purified from the culture supernatant by protein G and protein A affinity chromatography.

Synthesis of Antibody-Peptide Conjugate.

T15H peptide ASRNKANDYTTDYSASVKGRFIVSR (SEQ ID NO. 1), a VH-derived peptide from an autophilic antibody-T15, was synthesized by Genemed Synthesis (San Francisco, Calif., U.S.A.). Antibodies were dialyzed against PBS (pH 6.0) and 1/10 volume of 200 μmol/L sodium periodate was added and incubated at 4° C. for 30 minutes in the dark. The reaction was stopped by adding glycerol to a concentration of 30 μmol/L, and the sample was dialyzed at 4° C. for 30 minutes against PBS (pH 7.0). A one hundred times molar excess of T15H or scrambled T15 peptide (T15scr/T15s) SYSASRFRKNGSIRAVEATTDVNSAYAK (SEQ ID NO: 3) was added to the antibodies and incubated at 37° C. for 1 hour. L-Lysine was added and incubated at 37° C. for 30 minutes to block the remaining aldehyde group. The same oxidation reaction (except adding the peptides) was applied to antibodies used as controls. After the blocking step, the antibody conjugates were dialyzed against PBS (pH 7.2) overnight.

Ig Capture ELISA.

Four μg/mL of murine S1C5-T15H was coated to Costar vinyl assay plates (Costar, Cambridge, Mass.). After blocking with 3% BSA solution, 8 μg/mL of photobiotinylated S1C5-T15H, S1C5-scrambled peptide conjugate, and control S1C5 were added to the first wells, and 1:1 dilution was performed. The antibodies were incubated for 2 hours at room temperature. After washing with PBS buffer, avidin-HRP (Sigma-Aldrich, St. Louis, Mo.) was added as a 1:2500 dilution. The binding antibodies were visualized by adding substrate o-phenylenediamine.

Size Exclusion Chromatography.

Antibody conjugate was chromatographed on a 75 mL Sephacryl 300HR column (Pharmacia, Peapack, N.J.). 1:10 diluted PBS (pH 7.2) was chosen as elution buffer. Fractions (0.5 mL/each) were collected and aliquots (100 μL) were assayed on antihuman IgG capture ELISA. The ELISA reading (OD 490 nm) is plotted against elution volume.

Viability Assay for Antibody-Treated Cells.

Lymphoma cells were grown in 96-well tissue culture wells in 1-mL medium. Two μg of antibodies or antibody-peptide conjugates were added and incubated for various times as described herein. Ten μL aliquots from the cell suspension were used to determine viability by using trypan blue exclusion.

FACS Assay of the B-Cell Lymphoma.

Human Su-DHL4 and murine 38C13 cells were fixed with 1% paraformaldehyde. 1×10⁶ cells were suspended in 50 μL of staining buffer (Hank's balanced salt solution, containing 0.1% NaN₃, 1.0% BSA), then 1.5 μg of photobiotinylated murine S1C5-T15H conjugates was added and incubated for 30 minutes on ice. Control antibodies and antibody-scrambled T15 peptide conjugates served as controls. The cells were washed twice with staining buffer before avidin-FITC (Sigma-Aldrich) was added to the cells for 30 minutes on ice. Then the cells were washed twice with staining buffer, re-suspended in 200 μL PBS and analyzed by flow cytometry.

Hoechst-Merocyanin 540 Staining to Detect Apoptosis.

1×10⁶ of lymphoma cells were placed into 24-well tissue culture wells. Four μg of antibodies or antibody-peptide conjugates were added and incubated for various times as described herein. 1×10⁶ cells were removed from the culture, re-suspended in 900 μL cold PBS (pH 7.2). One hundred μL of Hoechst 33342 (50 μg/mL; Molecular Probe, Eugene, Oreg., U.S.A.) was added, the cells were incubated at 37° C. for 30 minutes in the dark. The cells were centrifuged and re-suspended in 100 μL PBS. Then, 4 μL of MC540 solution (Molecular Probe) was added, and 20-minute incubation was performed at room temperature in the dark. The cells were pelleted, re-suspended in 1 mL cold PBS (pH 7.2), and analyzed by flow cytometry.

Results

Characterization of Autophilic Antibodies.

The T15H (24-mer) peptide was crosslinked to two murine mAb (S1C5 and 5D10), using carbohydrate periodate conjugation. The mAb S1C5 (IgG1) is specific for the tumor idiotype of the mouse 38C13 B-cell line and the 5D10 antibody for the human Su-DHL4 B-cell tumor. Both mAbs recognize unique idiotypes of the BCR IgM on the B-cell tumors.

Autophilic Behavior Can Easily be Demonstrated by ELISA.

The autophilic effect was studied with the S1C5-T15H Mab conjugate. The T1SH-crosslinked S1C5 binds to insolubilized S1C5-T15H detected by biotin-avidin ELISA. Control S1C5 does not bind significantly to S1C5-T15H or S1C5 crosslinked with a scrambled peptide. Similar self-binding of T15H peptide-crosslinked mAb 5D10 to insolubilized T15H-5D10 was also observed. The specificity of the peptide mediated autophilic effect was tested using the 24-mer peptide T15H itself as an inhibitor. Only the T15H peptide inhibited SIC5-T15H and 5D10-T15H self-binding while the control-scrambled peptide did not inhibit it. These results are similar to previous inhibition data with the naturally occurring autophilic T15/S107 antibody (Halpern, R., et al., 1991).

T15H-Antibody Conjugates in Monomer-Dimer Equilibrium in Solution.

The non-covalent nature of the self-aggregation of T15H-linked antibodies raises the question of its physical state in solution. To address this issue, the molecular species of T15H-linked monoclonal antibodies were analyzed using gel electrophoresis and sizing gel filtration. The electrophoretic mobility of control and T15H peptide conjugated to S1C5 and 5D10 under reducing and non-reducing conditions show no differences, indicating the absence of chemical bonds between the antibody chains. The molecular species of the peptide-conjugated antibodies (5D10-T15H) was further analyzed by size exclusion chromatography. The elution profile indicated two immunoglobulin species of different sizes. The larger first peak eluted in the position of an antibody dimer. The second smaller peak eluted in the position of non-conjugated 5D10 antibody. The appearance of two peaks resembled monomer and dimer antibodies and could indicate that either a fraction of antibodies was not modified, or that the modification was complete and the antibody establishes an equilibrium of dimers and monomers. To test the latter possibility, material from both peaks were subjected to a second gel filtration on the same column. Reruns of both peaks yielded again two peaks at the same position as in the first chromatography (Zhao and Kohler, 2002). These data show that the T15H peptide-linked antibodies exist in solution as two distinct molecular species in equilibrium as monomer and dimer.

Enhanced Binding of Autophilic Antibodies to Tumors.

The binding of the peptide-conjugated antibodies against their respective tumor targets was compared with that of the control antibodies in indirect fluorescence activated cell sorting (FACS). As control, antibodies linked with a scrambled peptide were included. The fluorescence intensity of the T15H-S1C5 on 38C13 cells is compared with that of the control S1C5 and the scrambled peptide S1C5. The difference in mean fluorescence channels between S1C5-T15H and controls was greater than 10-fold. Similarly, the FACS analysis of autophilic 5D10-T15H on Su-DHL4 cells shows enhancement of binding over binding of control 5D10 and control peptide-crosslinked 5D10. In both tumor systems, the conjugation of the T15 H peptide to tumor-specific antibody enhanced the FACS signals over control antibodies used at the same concentration (Zhao, Lou, et al., 2002). The enhancement of fluorescence can be explained with the increase of targeting antibodies caused by self-aggregation and lattice formation on the surface of the tumor cells.

Inhibition of Tumor Growth.

Antibodies binding to the BCR induce crosslinking of the BCR, which, in turn, inhibits cell proliferation and produces a death signal. Furthermore, chemically dimerized antibodies directed against a B-cell tumor induce hyper-crosslinking of the BCR followed by inhibition of cell division and apoptosis of the tumor. To see if similar enhancement of the antitumor effects of dimerizing antibody were induced by noncovalent, dimerizing T15H-linked antibodies, the two B cell tumors were cultured in the absence or presence of control and T15H-linked antibodies. Co-culture of both tumors, 38C13 and Su-DHL4, with their respective T15H-linked antibodies inhibited the cell growth significantly better compared with the control antibodies. To test the tumor target specificity of autophilic antibodies in growth inhibition, criss-cross experiments were performed with the 38C13 and Su-DHL-4 cell lines. Inhibition of murine 38C13 cell growth with S1C5-T15H was statistically greater than mismatched 5D10-T15H. Similar results on the specificity of autophilic antibodies were obtained with the Su-DHL4 cells (Zhao, Y., et al., 2002).

Induction of Apoptosis.

As suggested by earlier studies, the antitumor effect of antibodies directed against the BCR of B-cell lymphomas in vitro and in vivo might be caused by the induction of apoptosis. Aliquots of tumor cells (38C13 and Su-DHL-4) cultured in the presence of control or T15H-linked antibodies were analyzed for apoptosis using a double stain FACS protocol. 38C13 and Su-DHL4 cells underwent a moderate amount of apoptosis without antibodies over a 6, respectively, 18-hour culture. This apoptosis was enhanced when the respective antibody was added. However, when the T15H-linked antibodies were added, the accumulated number of apoptotic 38C13 cells was almost doubled, and apoptosis of Su-DHL4 cells was more than doubled during the entire culture (Zhao, Y., et al., 2002).

Discussion

The biologic advantage of the autophilic property is exemplified with the S107/T15 anti-phosphorylcholine antibody. This autophilic antibody is several times more potent in protecting immune-deficient mice against infection with Pneumococci pneumoniae than non-autophilic antibodies with the same antigen specificity and affinity.

As shown here, the autophilic antibody function can be transferred to other antibodies by chemically crosslinking a peptide derived from the T15 VH germline sequence. The modified antibody mimics the autophilic property of the T15/S107 antibody, producing an autophilic antibody with increased avidity and enhanced targeting. Enhancing the binding of autophilic engineered antibodies to the BCR of B-cell tumor increases the strength of the death signals leading to profound inhibition of cell proliferation in culture. Even though a doubling of apoptosis is demonstrated here, other mechanisms of growth inhibition can be involved.

Crosslinking the BCR of the mature murine B-cell lymphoma A20 can protect against CD95 mediated apoptosis. This anti-apoptotic activity of engagement of the BCR by crosslinking antibodies is highly restricted to the time window of CD95 stimulation and is not dependent upon protein synthesis. The finding that BCR hypercrosslinking per se is pro-apoptotic is not at variance with reports on the anti-apoptotic activity of the BCR engagement, because it can be due to the use of less mature B-cell lines, to different strength of delivered signals by homodimerizing antibodies, or to Fas-independent apoptosis.

The use of two BCR idiotope-specific antibodies against different tumors offered the opportunity to test the biologic effect of targeting receptors other than the idiotope specific BCR. In criss-cross experiments with autophilic antibodies binding in FACS analysis and inhibition of growth in vitro show a significant enhancement only with the autophilic matched antibody. In this context, it is interesting to speculate whether enhanced tumor targeting would also augment cellular effector functions.

In an earlier study using chemically homodimerized antibodies, the Fc domain was not involved in the augmentation of growth inhibition and tumor cells lacking Fc receptors were susceptible to the antigrowth activity of homodimers. Thus, the anti-tumor effect induced by dimerizing antibodies would not be restricted to lymphoid tumors such as non-Hodgkin's B-cell lymphoma, where anti-tumor effects require the participation of Fc-receptor-bearing effector cells.

The described approach of transferring the naturally occurring autophilic property to other antibodies thereby enhancing their anti-tumor effect outlines a general method to improve the therapeutic efficacy of antibodies in passive immunotherapy. Such noncovalent antibody complexes offer several advantages over chemically crosslinked antibodies: (i) the equilibrium between monomer and noncovalent homopolymers prevents the formation of precipitating nonphysiologic complexes in solution; (ii) autophilic conversion does not compromise the structural integrity of antibodies; and (iii) the method is simple and efficient and does not require a purification step typically needed for chemically crosslinked homodimers that reduces the yield of active Ig dimers. One possible limitation of the approach of using dimerizing antibodies might be the ability to penetrate a large tumor mass. Because the homophilic peptide is of murine origin, it might be immunogenic in humans. Thus, it could be necessary to humanize the murine peptide based on sequence and structural homology using computer modeling. The demonstration that adding a single peptide to the structure of antibodies increases the amount of antibody bound to targets and the anti-tumor activity encourages attempts to engineer recombinant antibodies expressing the autophilic activity.

### Example 2

### Internalization of Antibodies Conjugated with MTS Peptide

Cell Line and Antibodies

Human Jurkat T cells were grown in RPMI 1640 supplemented with 10% fetal bovine serum and antibiotic (penicillin, streptomycin and amphotericin). Rabbit polyclonal anti-active caspase-3 antibody (#9661 S) and anti cleaved-fodrin, i.e., alpha II spectrins (#2121S), were purchased from Cell Signaling, Inc (Beverly, Mass.). Monoclonal (rabbit) anti-active caspase-3 antibody (#C92-605) was purchased from BD PharMingen (San Diego, Calif.). Mouse monoclonal antibody 3H1 (anti-CEA) was purified from cell-culture supernatant by protein G affinity chromatography. Anti-mouse and anti-rabbit HRP-conjugated secondary antibodies were purchased from Santa Cruz Biotechnologies, Inc. ApoAlert Caspase-3 Fluorescent Assay kit was purchased from Clontech Laboratories (Palo Alto, Calif.). The Cell Death Detection ELISA was purchased from Roche Applied Science (Indianapolis, 1N).

Synthesis of MTS Peptide-Antibody Conjugate

MTS peptide KGEGAAVLLPVLLAAPG (SEQ ID NO. 2) is a signal peptide-based membrane translocation sequence, and was synthesized by Genemed Synthesis (San Francisco, Calif.). Antibodies were dialyzed against PBS (pH 6.0) buffer, oxidized by adding 1/10 volume of 200 mmol/L NaIO₄ and incubating at 4° C. for 30 min in the dark. Adding glycerol to a final concentration of 30 mM terminated the oxidation step. Samples were subsequently dialyzed at 4° C. for 1 h against 1×PBS (pH 6.0) buffer. The MTS peptide (50× molar excess) was added to couple the antibodies and the samples were incubated at 37° C. for 1 hour and the resulting antibody-peptide conjugate was dialyzed against 1×PBS (pH 7.4).

Effect of MTS-Conjugated Antibody on Cell Growth

Jurkat cells (2.5×10⁵) were seeded into 96-well culture plate. After incubation with 0.5 μg MTS-antibody conjugates for 6, 12, 18 and 24 hour, aliquots were removed and viability was determined by trypan blue exclusion.

Study of Antibody Internalization by ELISA

Jurkat cells, grown in 1-ml medium in a 6-well culture plate, were incubated with 2 μg of unconjugated or MTS conjugated antibodies for 0, 1, 3, 6, 12 and 18 h. The cells were centrifuged and the culture supernatant was then transferred to a new tube. The cell pellet was washed twice with PBS (pH 7.4) before being homogenized by Pellet Pestle Motor (Kontes, Vineland, N.J.) for 30 sec. All of the cell homogenate and an equal volume of the culture (10 μl) supernatant were added to sheep anti-rabbit IgG coated ELISA plate (Falcon, Oxnard, CA) and incubated for 2 h at room temperature. After washing, HRP-labeled goat anti-rabbit light chain antibody was added, and visualized using o-phenylenediamine.

### DNA Fragmentation

Jurkat cells were pre-treated with antibodies or a caspase-3 inhibitor (DEVD-fink) for 1 h, centrifuged, and incubated with fresh medium containing actinomycin D alone (1 μg/ml) for 4 h. After treatment, Jurkat cells were collected, washed, and resuspended in 700 μl of HL buffer (10 mM Tris-HCl, pH 8.0, 1 mM EDTA, 0.2% Triton X-100, for 15 min at room temperature. DNA was extracted with phenol:chloroform:isoamyl alcohol (25:24:1) and precipitated 24h at −20° C. with 0.1 volume of 5 M NaCl and 1 volumes of isopropanol. The DNA was washed, dried, and resuspended in TE pH 8.0. The DNA was resolved by electrophoresis on a 1.5% agarose gel and visualized by UV fluorescence after staining with ethidium bromide. DNA fragmentation was also determined using the Cell Death Detection ELISA according to the manufacturer's instructions.

Preparation of Total Cell Lysate

Jurkat cells were treated as described in the DNA fragmentation section. After treatment, cells were collected and washed with PBS (pH 7.4) twice, then suspended in 3001 of CHAPS buffer (50 mM PIPES, pH 6.5, 2 mM EDTA, 0.1% CHAPS). The samples were sonicated for 10 sec and centrifuged at 14,000 rpm for 15 min at 4° C. The supernatant was transferred to a new tube and referred as total cell lysate.

Caspase-3-Like Cleavage Activity Assay

Jurkat cells were treated as described in the DNA fragmentation section. Equal amounts of protein of the total cell lysate were applied for caspase-3 activity assay using ApoAlert Caspase-3 Fluorescent Assay Kit according to the manufacturer's instruction. Fluorescence was measured with a Spectra MAX GEMINI Reader (Molecular Devices, Sunnyvale, Calif.).

Western Blot Analysis

Jurkat total cell lysates (10 μg) were separated on a 10% SDS-PAGE gel to detect immunoreactive protein against cleaved spectrin. Ponceau staining was used to monitor the uniformity of protein transfer onto the nitrocellulose membrane. The membrane was washed with distilled water to remove excess stain and blocked in Blotto (5% milk, 10 mm Tris-HCl [pH 8.0], 150 mM NaCl and 0.05% Tween 20) for 2 h at room temperature. Before adding the secondary antibody, the membrane was washed twice with TBST (10 mM Tris-HCl with 150 mM NaCl and 0.05% Tween 20), and then incubated with HRP-conjugated secondary antibodies. The blot was washed extensively and reactivity was visualized by enhanced chemiluminescence (AmershamBiotech, Piscataway, N.J.).

Statistical Analysis

Statistical analysis was performed using the student t-test (for a pair-wise comparison) and one-way ANOVA followed by Newman-Keuls posttest. Data are reported as means±SE.

Results

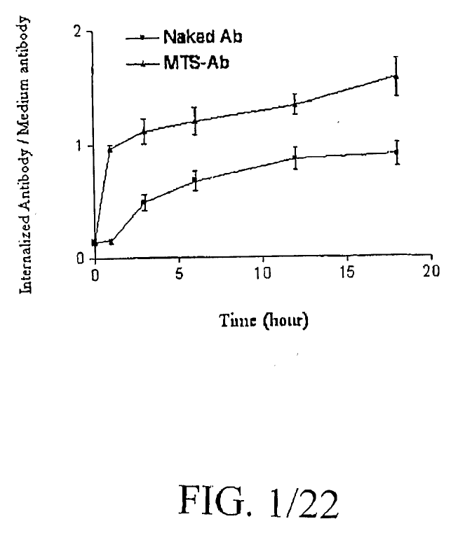

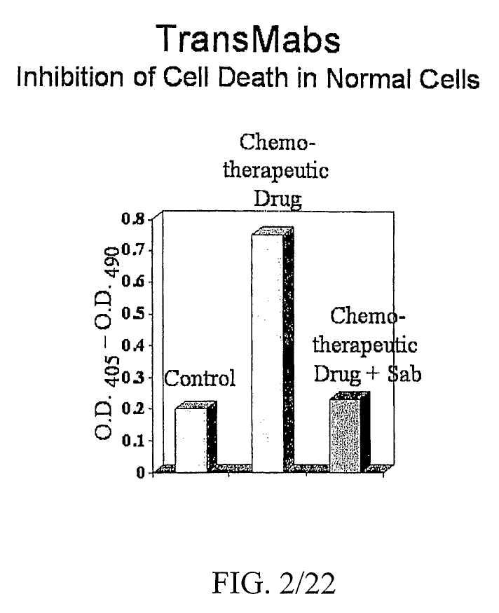

As shown in FIG. 1, an MTS conjugated anti-active caspase 3 antibody is internalized more rapidly than unmodified antibody. When cells were exposed to the chemotherapeutic drug, actinomycin D, apoptosis was triggered and the cells died (see FIG. 2). However, if cells were exposed at the same time to the MTS-conjugated antibody (transMab), most of the toxicity of the chemotherapeutic drug was inhibited.

### Example 3

### Enhancing Binding and Apoptosis Using Peptide-Conjugated Anti-CD20 Antibodies

Cell Line and Antibodies

The human B-cell tumor lines SU-DHL-4 and Raj were grown in RPMI 1640 medium, supplemented with 10% fetal bovine serum, 2 mmol/L glutamine, 10 μmol/L Hepes, 50 U/mL penicillin, 50 μg/mL streptomycin, and 50 μmol/L 2-mercaptoethanol at 37° C. under 5% carbon dioxide. Mouse monoclonal antibodies 1F5 IgG2a (ATTC #HB-9645) specific for human B-cell lymphomas 5D10 and 3H1 (Zhao, Lou, et al., 2002.) were purified from cell culture supernatant by protein G or protein A affinity chromatography.

Synthesis of Antibody-Peptide Conjugate

T15 peptide ASRNKANDYTTDYSASVKGRFIVSR (SEQ ID NO. 1), a VH-derived peptide from a self-binding antibody-T15 was synthesized as described in Example 1. 8-azido-adenosine-biotin was synthesized and used to affinity cross-link biotin to antibodies. The 8-azidoadenosine dialdehyde was prepared as previously described (U.S. Pat. No. 5,800,991, issued to Haley et al., which is incorporated herein by reference).

Self-Binding Enzyme-Linked Immunosorbent Assay

Four micrograms per milliliter of 1F5-T15 was used to coat Costar vinyl assay plates (Costar, Cambridge, Mass., U.S.A.). After blocking with 1% BSA solution, 8 μg/mL photobiotinylated 1F5-T15 naked 1F5 and control antibody (5D 10) were added, diluted to 1:1, and incubated for 2 hours at room temperature. After washing with PBS buffer, avidin-HRP (Sigma-Aldrich) was added, and enzyme-linked immunosorbent assay color was developed with o-phenylenediamine.

FACS Assay of the B-Cell Lymphoma

SU-DHL-4 cells were fixed using 1% paraformaldehyde, and 1×10⁶ cells were suspended in 50 μL staining buffer (Hanks, containing 0.1% NaN3 and 1.0% BSA); 1.5 μg photobiotinylated 1F5-T15 conjugates, naked IF5, and control antibodies were added and incubated for 30 minutes on ice. The cells were washed twice with staining buffer, and then avidin-FITC was added for 30 minutes on ice. After washing twice with staining buffer, the cells were resuspended in 200 μL PBS for FACS analysis.

Hoechst-Merocyanin 540 Staining to Detect Apoptosis

After 1×10⁶ lymphoma cells were placed into 24-well tissue culture wells, 4 μg antibodies and antibody-peptide conjugates were added. After 24 hours of incubation, 1×10⁶ cells were removed from the culture pellet and resuspended in 900 μL cold PBS (pH 7.2), and 100 μL Hoechst (Pierce, Rockford, Ill., U.S.A.) 33342 (50 g/mL) was added and incubated at 37° C. for 30 minutes in the dark. The cells were centrifuged and resuspended in 100 μL PBS; 4 μL MC540 dilution solution was added and the cells were incubated for 20 minutes at room temperature in the dark. The cells were pelleted, resuspended in 1 mL PBS, and analyzed by flow cytometry.

Inhibition of Cell Growth in Culture

1×10⁵ tumor cells were seeded in complete culture medium. At days 1, 2, and 3 of culture, aliquots were removed and viable cells were counted (trypan blue).

Results

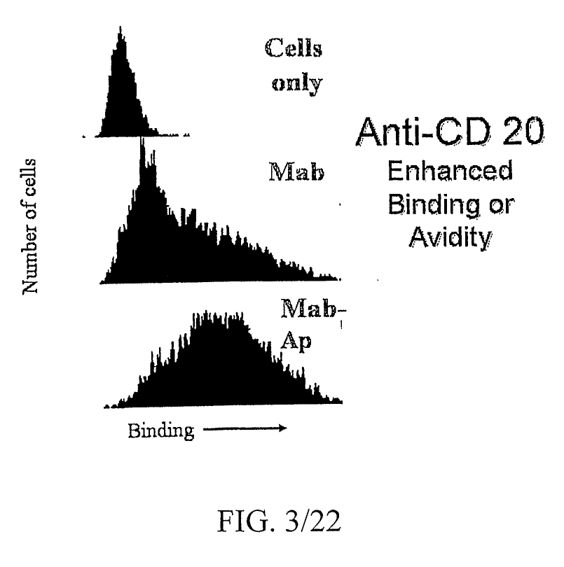

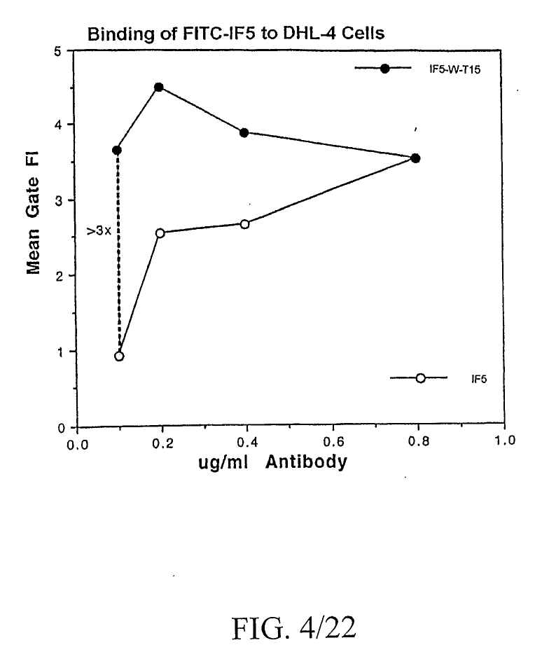

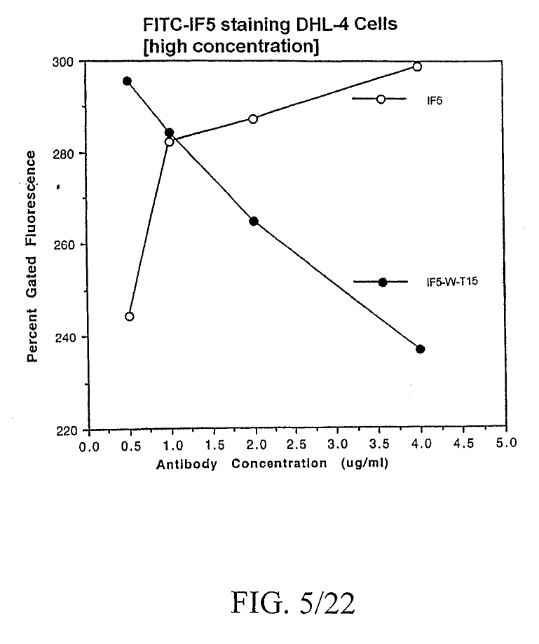

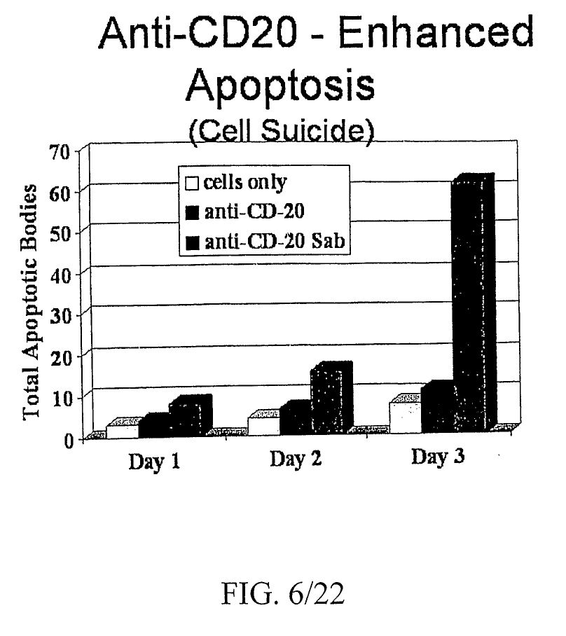

Mouse monoclonal antibodies 1F5 IgG2a were conjugated with self-binding peptide as in Example 1. An average of 1.8 peptides per antibody was found by competitive analysis. The parental antibody was compared to the conjugated form for binding by flow cytometry. As shown in FIG. 3, the binding was increased for the conjugated antibody (Mab-ap) when assessed with a limiting dilution of antibody. This was characterized by a shift in the binding fluorescence to a higher intensity. When compared over a series of dilutions, conjugated antibody required almost one-tenth the concentration of antibody to achieve the same level of intensity as parental antibody (FIG. 4). As shown in FIG. 5, increasing the amount of conjugated antibody caused a reduction in fluorescence intensity, presumably due to internalization, a property of SAT technology that can be used to enhance potency of immunoconjugates of drugs, toxins and short path length radiotherapeutic isotopes. Furthermore, when tested for the ability to trigger apoptosis, the conjugated form (Sab) was much more active than native antibody, with most cells dead by 3 days, compared to only a small fraction with the native antibody (FIG. 6).

### Example 4

### Enhanced Binding and Apoptosis with Anti-GM2 Antibodies

Cell Lines and Antibody

Human T-cell leukemia Jurkat cells were grown in RPMI 1640 supplemented with 10% fetal bovine serum and antibiotic (penicillin, streptomycin and amphotericin). Chimeric hamster anti-GM2 antibody (ch-α-GM2) was obtained from Corixa Corporation (Seattle, Wash.). After chimerization, the resulting antibody lost its ability to induce apoptosis in ganglioside GM2 expressing target cells.

Synthesis of Antibody-Peptide Conjugate

Both T15 peptide ASRNKANDYTTEYSASVKGRFIVSR (SEQ ID NO: 1), a VH-derived peptide from a self-binding antibody-T15 (Kaveri et al, 1991), and a scrambled T15 peptide (T15-scr) (SEQ. ID. NO. 3), randomly generated from the T15 amino acid sequence, were synthesized by Genemed Synthesis (South San Francisco, Calif.). The scrambled peptide was used as a control. Antibodies were dialyzed against PBS (pH 6.0), then 1/10 volume of 200 μM NaIO₄ was added and incubated at 4° C. for 30 min in the dark. The reaction was stopped by adding glycerol to a final concentration of 30 μM, and the samples were dialyzed at 4° C. for 30 min against PBS (pH 6.0). Fifty (50) times molecular excess of T15 or scrambled peptide was added to the antibodies and incubated at 37° C. for 1 h. L-Lysine was added and incubated at 37° C. for 30 min to block the remaining reactive aldehyde group. After the blocking step, the antibody-conjugates were dialyzed against PBS (pH 7.2) at 4° C. overnight, then stored at 4° C. until used.

Direct Binding ELISA

GM2 ganglioside was dissolved in methanol and 0.5 μg was coated per well in 96 well polystyrene plates (Costar, Cambridge, Mass.) and allowed to dry overnight. The wells were blocked with 1% BSA for 2 h at room temperature and 400 μg of anti-GM2 antibodies, diluted in 1% BSA, were added in the first well and then serially diluted 1:1. After incubation for 1 h, the wells were washed 5× and HRP-conjugated anti-human IgG (Sigma-Aldrich) was added at a 1:1000 dilution and incubated for 1.5 h. After washing three times, the bound antibodies were visualized using substrate o-phenylenediamine and read at OD 492 using a spectrophotometer.

Specific Binding ELISA

Gangliosides GM2, GM1, GM3 were dissolved in DMSO in 0.5 μg and coated in a 96 well polystyrene plate (Costar, Cambridge, Mass.) dried overnight. The wells were blocked with 1% BSA for 2 h at room temperature, 400 μg of ch-α-GM2 antibodies (anti-GM2-T15) were added in the first well and then serially diluted 1:1. After incubation for 1 h, the wells were washed 5 times and HRP-conjugated anti-human IgG was added and incubated for 1.5 h. After washing three times, the bound antibodies were visualized using substrate o-phenylenediamine and assayed as described previously.

Antibody Self-Binding ELISA

2 μg/ml of naked ch-α-GM2 (anti-GM2) or ch-α-GM2-T15 (anti-GM2-T15) were coated onto Costar vinyl assay plates. After blocking with 3% BSA solution, 0.5 μg/well of photobiotinylated anti-GM2-T15 was added. The antibodies were then incubated for 2 h at room temperature. After washing three times, avidin-HRP (Sigma-Aldrich) was added at a 1:1000 dilution and incubated for 1 hour. The bound antibodies were visualized with o-phenylenediamine and assayed as described previously.

Cell Surface Binding Detected by Facs

2×10⁵ Jurkat cells per well were seeded in a 6-well plate and incubated overnight, then cells were collected and washed twice with P/B/G/A buffer (0.5% BSA, 5% Goat Serum in PBS). Cells were then resuspended in 100 μL P/B/G/A buffer containing 5 μg/ml anti-GM2 antibodies for 30 min. After washing with P/B/G/A buffer, FITC-conjugated anti-Human IgG (Sigma-Aldrich, 1:1000 dilution in 100 μL P/B/G/A) was added and incubated on ice for 30 min. After washing with P/B/G/A buffer, cells were resuspended in 400 μL P/B/G/A containing 10 μg/ml propidium iodide (as viability probe) and analyzed by flow cytometry.

Apoptosis Detected by Annexin V Staining

2×10⁵ Jurkat cells were seeded per well in a 6-well plate. After 6 h, cells were incubated with 20 μg/ml of the anti-GM2 or anti-GM2-T15 antibodies for 12 hr. Following the incubation, a small portion of cells (50 μL) was saved and assayed for viability, while the remainder of the cells were harvested and washed with cold PBS. Cells were then resuspended in 100 μL annexin staining buffer, 5 μL Alex fluor 488 was added into 95 μL 1× annexin binding buffer, and Sytox was added at a dilution of 1:1000. After incubation at room temperature for 15 min, 400 μL of 1× annexin binding buffer was then added, and samples were analyzed by FACS.

Viability Assay for Antibody-Treated Cells

A small portion of the cell samples saved from the annexin experiment was used for viability assay. 10-μL aliquots from the cell suspension were taken to determine viability using trypan blue exclusion assay.

Statistical Analysis.

Statistical analysis was performed using one-way ANOVA followed by Newman-Keuls post test. Data are reported as means±SD.

Results

Self-Binding Peptide Enhanced Antibody Binding to its Specific Ganglioside.

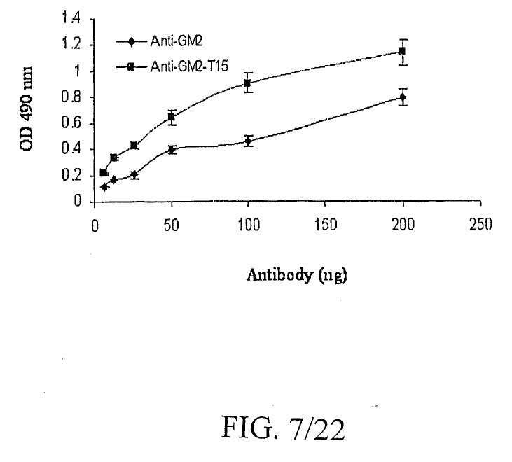

Following antibody-peptide conjugation, the binding capacity of the T15-conjugated ch-α-GM2 antibody (anti-GM2-T15) was determined using a direct binding ELISA. As seen in FIG. 7, both ch-α-GM2 antibody (anti-GM2) and anti-GM2-T15 antibody showed a dose-dependent increase in binding to ganglioside GM2. The anti-GM2-T15 antibody demonstrated a higher binding capacity compared with the naked anti-GM2 at all the doses tested, confirming that the self-binding T15 peptide had increased the antigen binding capacity of the ch-α-GM2 antibody at a given antibody concentration.

Antibody Self-Binding Behavior Demonstrated by ELISA

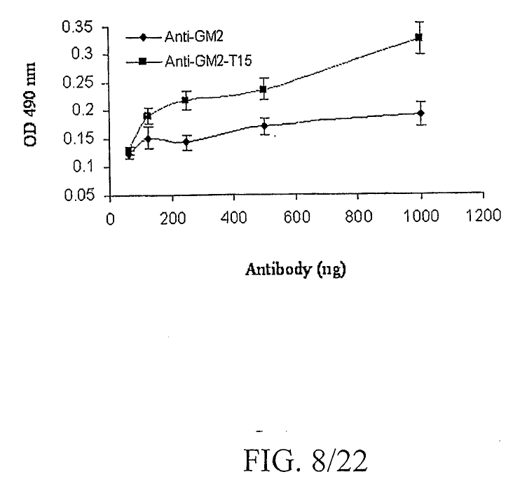

Next, it was investigated by ELISA whether the increase in binding to ganglioside GM2 by the T15 peptide-linked antibody was due to its self-binding feature. As seen in FIG. 8, the anti-GM2-T15 antibody demonstrated a greater dose-dependent increase in binding to the peptide-conjugated anti-GM2-T15 antibody coated on the wells, whereas it did not show significant binding to the non-peptide conjugated anti-GM2 antibody. These data demonstrate that the anti-GM2-T15 antibody can bind to itself or homodimerize through the Fc-conjugated, autophilic peptide moiety.

T15 Conjugation does not Change the Specificity of the Ch-α-(Gm2 Antibody.

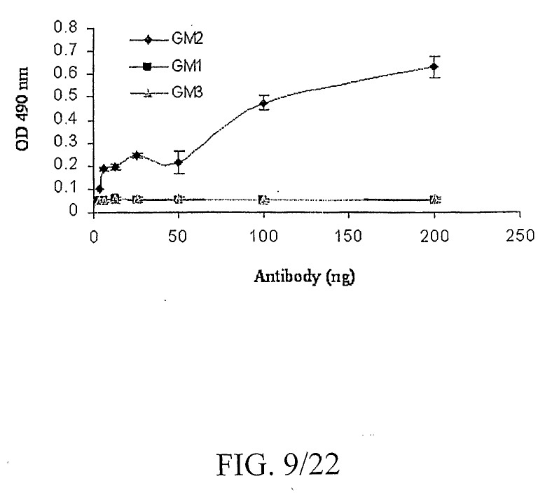

To assess whether conjugation of the T15 peptide might alter the cognate binding specificity of the antibody, a direct antigen-binding ELISA was used to determine the binding specificity of the anti-GM2-T15 conjugated antibody. As shown in FIG. 9, the anti-GM2-T15 antibody demonstrated a specific, dose-dependent increase in binding to ganglioside GM2, whereas no binding above background levels to gangliosides GM1 or GM3 was detected. This result confirms that addition of the self-binding T15 peptide did not alter nor reduce the specificity of the ch-α-GM2 antibody.

Enhanced Surface Binding of Anti-Gm2 Antibody to Target Tumor Cells

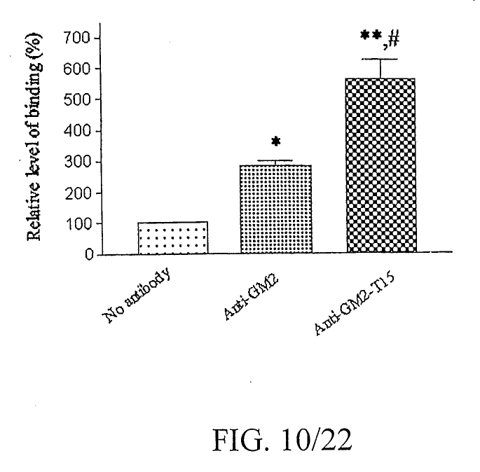

The human T-cell leukemic cell line Jurkat is known to express ganglioside GM2 (Suzuki et al, 1987). The ability of the peptide-conjugated anti-GM2-T15 antibody to bind to native ganglioside GM2 expressed on the surface of Jurkat cells was compared to that of the non-conjugated anti-GM2 antibody by flow cytometry. As shown in FIG. 10, the ch-α-GM2 antibody (anti-GM2) demonstrated a GM2 specific binding signal three times greater than background levels, whereas the binding demonstrated by the T15-conjugated anti-GM2 antibody was 2-fold higher than that of the non-peptide conjugated antibody. This result suggests that the enhanced binding demonstrated by the peptide-conjugated Ab is due to self-aggregation of this antibody.

Inhibition of Tumor Growth

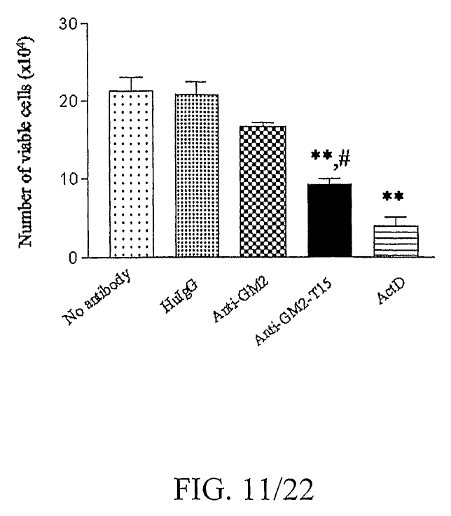

Antibodies binding to the B cell receptor have been shown to induce crosslinking of the BCR, which, in turn, inhibits cell proliferation (Ward et al, 1988) and produces a death signal (Hasbold et al, 1990; Wallen-Ohman et al, 1993). Furthermore, chemically dimerized antibodies directed against a B-cell tumor induce hyper-crosslinking of the BCR followed by inhibition of cell division and induction of apoptosis of the tumor cells (Ghetie et al, 1994; Ghetie et al, 1997). To determine whether the T15-conjugated anti-GM2 antibody induced a similar anti-proliferative effect, 2×10⁵ Jurkat cells were cultured in the presence or absence of anti-GM2 or control antibodies for 12 h, and then the number of viable cells remaining was counted. As summarized in FIG. 11, “no antibody” or control human IgG antibody (HuIgG) treatment had no effect on cell growth or viability, whereas there was some effect with the anti-GM2 antibody. However, the T15-linked antibody demonstrated a marked inhibition of Jurkat cell growth, as cell numbers were reduced>2-fold compared to naked anti-GM2 antibody treated cells, and more than 4 fold versus the control IgG treatment. As a comparison and positive control, Actinomycin D demonstrated the ability to induce apoptosis, at levels slightly higher than the SuperAntibody.

Induction of Apoptosis

In order to determine whether the anti-tumor effect of antibodies directed against cell surface expressed gangliosides might be due to the induction of apoptosis, the cell samples used in the cell growth study were analyzed for apoptosis induction by measuring annexin V staining. The results are summarized in Table 2.

TABLE 2
Apoptosis analysis using Annexin V staining.

| Antibody | Jurkat* |
|---|---|
| No treatment | 7.7 ± 1.55 |
| HuIgG | 7.2 ± 1.94 |
| Anti-GM2 | 14.8 ± 7.55 |
| Anti-GM2-T15scr | 13.0 ± 4.60 |
| Anti-GM2-T15 | 54.2 ± 23.4 |
| Actinomycin D | 81.9 ± 10.2 |

*Data were summarized from four sets of experiments.

Treatment of Jurkat cells with the ch-α-GM2 antibody (anti-GM2) or the ch-α-GM2 antibody conjugated with a scrambled, control peptide (anti-GM2-T15scr) did not induce apoptosis significantly over levels induced by treatment with control human IgG, as a modest 2-fold increase was observed. However, Jurkat cells treated with the anti-GM2-T15 conjugated underwent a significant amount of apoptosis, nearly 8-fold over background and more than 4-fold higher than that induced by the non-conjugated antibody or the control-conjugated antibody. These results confirmed the activity and specificity of T15-conjugated antibody.

### Example 5

### Generation of Autophilic Peptide Sequences T15-scr, T15-scr2 R24, and R24—Charged

Peptides were synthesized as in Example 1. The sequences are given in Tables 3 and 4.

TABLE 3
Sequences for Autophilic Binding Peptides

| | | |
|---|---|---|
| Name | Sequence (NH2 to COOH) | SEQ ID NO |

| | | |
|---|---|---|
| T15 | ASRNKANDYTTDYSASVKGRFIVSR | 1 |
| T15scr or | SYSASRFRKNGSIRAVEATTDVNSAYAK | 3 |
| T15s | | |
| T15scr2 | SKAVSRFNAKGIRYSETNVDTYAS | 4 |
| R24 | GAAVAYISSGGSSINYA | 5 |
| R24-Charged | GKAVAYISSGGSSINYAE | 6 |
| T15 dipeptide | ASRNKANDYTTDYSASVKGRFIVS-gly-gly-gly-RR- | 10 |
| | gly-gly-gly-ASRNKANDYTTDYSASVKGRFIVS | |
| T15 tandem | ASRNKANDYTTDYSASVKGRFIVS-gly-gly-gly- | 11 |
| | ASRNKANDYTTDYSASVKGRFIVS | |

TABLE 4
Sequences for Membrane Penetrating Peptides

| | | |
|---|---|---|
| | | SEQ |
| | | ID |
| Name | Sequence (NH2 to COOH) | NO |

| | | |
|---|---|---|
| MTS | KGEGAAVLLPVLLAAPG | 2 |
| MTS-optimized | WKGESAAVILPVLIASPG | 7 |
| MTS dipeptide | KGEGAAVLLPVLLAAPG-gly-gly-gly-RR- | 12 |
| | gly-gly-gly-KGEGAAVLLPVLLAAPG | |
| MTS tandem | KGEGAAVLLPVLLAAPG-gly-gly-gly- | 13 |
| | KGEGAAVLLPVLLAAPG | |

The peptide derived from R24 is difficult to solubilize except in DMSO or alcohol. Using such solubilizers can not only denature the antibody but also makes it difficult to conjugate to hydrophilic regions of the antibody. To overcome this solubility problem the addition and changes of sequence to charged amino acids, as shown in Table 3, were undertaken. The resultant modified peptide (R24-Charged) was soluble in aqueous buffer, was able to be conjugated to the tryptophan or nucleotide binding sites and preserved self-binding as well as induced apoptosis when conjugated to anti-GM2 antibody. The same amino acids present in the T15 sequence were randomly re-arranged and used to construct a further synthetic peptide; this scrambled sequence (T15scr or T15s), had no self-binding and when conjugated to anti-GM2 antibody did not induce apoptosis (see Example 4, Table 2). In like manner, a second, randomly selected sequence, derived from the amino acids of the T15 sequence, was used to generate a synthetic peptide (T15scr2). Unlike the first scrambled sequence, this peptide demonstrated self-binding and when conjugated to anti-GM2 antibody, induced apoptosis in levels higher than the original T15 sequence. Thus, self-binding behavior can be generated, using the same amino acids from the original T15 sequence but arranged in a different order from the original T15. A peptide library generated using these same amino acids, combined with a screen for self-binding could be used to identify other self-binding sequences.

### Example 6

### Comparison Of Various Immunoglobulin Conjugation Sites

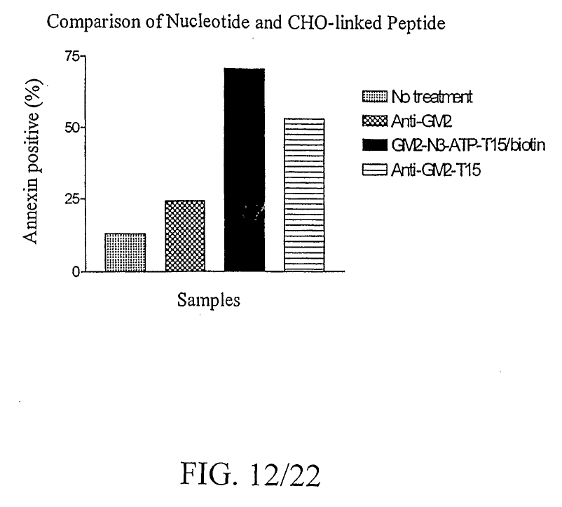

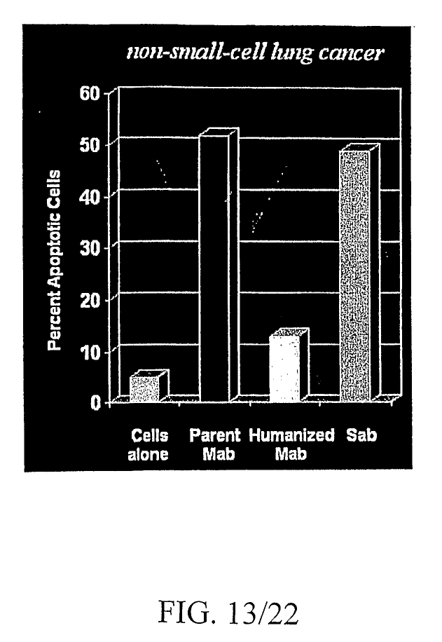

The T15 peptide sequence was conjugated to anti-GM2 antibody via the nucleotide binding site, tryptophan affinity sites, and through periodate oxidation of the carbohydrate on the Fc region. As shown in FIG. 12, when tested for the ability to trigger apoptosis, the nucleotide site conjugation (GM2-N3-ATP-T15/biotin) generated a higher level of apoptosis than the carbohydrate linkage (Anti-GM2-T15). This was in spite of the fact that carbohydrate linkage installed 8-10 peptides per antibody and nucleotide linkage only 2 peptides per antibody. Hence, affinity site conjugation was the best method of conjugation of peptides. Conjugation to epsilon-amino acids of antibody, via hetero-bifunctional cross-linking agents, gave an inactive conjugate (not shown).

### Example 7

### Restoration of Apoptotic Activity

A parental antibody to GM2 glycolipid, derived from a non-human hybridoma, was tested for the ability to trigger apoptosis against human cancers including non-small cell lung cancer (FIG. 13). The parental antibody demonstrated a high level of apoptosis and killing of cancer cells. The antibody was also effective in inhibiting growth of cancers in nude mouse models (not shown). To remove the potential for immunogenicity in humans, the antibody was “humanized” via cloning the heavy and light chain CDR's into the context of a human IgG1. Despite retention of affinity and specificity (not shown), the humanized antibody demonstrated much reduced ability to trigger apoptosis. In contrast, the humanized antibody, conjugated to a self-binding peptide (Sab), demonstrated high levels of apoptosis, similar to that of the parental antibody.

A further example is of a murine antibody, R24, which targets the GD3 ganglioside on human melanoma cells. When naturally expressed, this antibody has self-binding and therapeutic activity in patients, but as a humanized antibody it loses avidity, self-binding and therapeutic activity (Chapman et al., 1994). Restoration of therapeutic activity of the humanized R24 antibody can also be achieved by conjugation of a self-binding peptide to the antibody.

The humanized versions of antibody TEPC-15 and T15/S107 can also benefit from conjugation with a self-binding peptide to restore or enhance self-binding and therapeutic activity.

### Example 8

### Enhanced Binding and Tumor Recognition by Herceptin® SuperAntibody

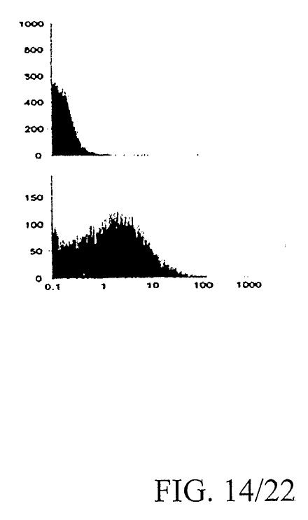

Herceptin® (monoclonal antibody to HER2/neu), has been approved by the FDA for treatment of breast cancer. The antigen is expressed in approximately 30% of breast cancers but in only about half of those patients is the level of expression sufficient to trigger therapeutic effects. In fact, patients are normally pre-screened in a diagnostic test to determine their suitability for treatment. HER2/neu is also expressed on other cancers, such as non-small cell lung cancer but typically in only low levels, making this type of cancer unsuitable for treatment. An autophilic peptide was conjugated to Herceptin and tested for ability to bind non-small cell lung cancer. As shown in FIG. 14 (top panel), Herceptin reacts very weakly to this cancer; only 0.5% of cells are positive compared to an irrelevant antibody. In contrast, the same cancer can be better detected with the autophilic peptide conjugated form (i.e., SuperAntibody form) of Herceptin; over 57% are positive compared to irrelevant antibody (bottom panel). In separate tests, a SuperAntibody form of Herceptin also inhibited growth better than the parent antibody and could trigger apoptosis unlike the parent.

### Example 9

### Photo-Crosslinking of Tryptophan Peptides to Antibodies

Antibodies and Reagents

Anti-human IgG (whole molecule)-peroxidase-conjugated secondary antibody, avidin-conjugated peroxidase, anti-human IgG (whole molecule) antibody, monoganglioside GM2 were purchased from Sigma-Aldrich. Anti-GM2 antibody, Herceptin and anti-GM3 were obtained from Corixa (Seattle, Wash.), Genentech (San Francisco, Calif.) and CMI (Havana, Cuba), respectively.

Two kinds of Trp-biotin peptides were designed: KAAGW (SEQ ID NO: 8) containing a biotin molecule on the alpha amino group [single biotin-peptide], and KAAKGEAKAAGW (SEQ ID NO: 9) containing biotin molecules on the alpha and epsilon amino groups of lysine [Multiple biotin-peptide]. These peptides were synthesized by Genemed Synthesis, Inc. (San Francisco, Calif.).

GM1, 2 and 3 were obtained from Sigma-Aldrich, glycolylic GM3 was obtained from Alexis USA (San Diego, Calif.).

Photobiotinylation Using the Tryptophan Site.

All antibodies were incubated with the tryptophan-containing peptides for 1 hr at room temperature. The antibodies were photo-biotinylated at 200, 100, 50, 25, 10 and 1 μM concentrations of biotin-peptide. Photo-crosslinking was done using UV crosslinker FP-UVXL-1000 (Fisher Scientific) on the optimum setting at 100 μj/cm². The samples were dialyzed against PBS (pH 7.4) buffer. The antibody concentration was determined using Comassie Plus Protein Assay (Pierce). Chemical biotinylation was performed with NHS-biotin (Pierce Chemical, Rockford, Ill.). Chimeric anti-GM3 glycolylic (CIMAB, Havana, Cuba) was biotinylated with 15 molar excess of NHS-biotin according to the manufacturer's protocol.

Direct Antibody Binding ELISA

Photobiotinylated antibody was coated by adding 2 μg to the first well and serially diluted and incubated overnight at 4° C. The wells are washed 3× and blocked with 3% BSA dissolved in PBS, pH 7.4 for 2 hours. The plate was washed 3× and 100 μL of a 1/1000 dilution of avidin peroxidase conjugate was added per well. After incubating for 1 hour at room temperature, the wells were washed 3× with washing solution. 100 μL of OPD solution (OPD buffer, o-phenylenediamine and 1 μL of 30% hydrogen peroxide per ml) were added to each well. The color development was stopped by adding 30 μL of 4N H₂SO₄ and the optical density is determined by scanning each well at 492 nm with a Fisher Scientific Multiskan RC plate reader.

Antibody Capture ELISA

Goat anti-human IgG whole molecule was coated at a 1/100 dilution per well, overnight at 4° C. The plate was washed 3× and blocked 2 hours at room temperature with 3% BSA in PBS, pH 7.4. The plate was washed 3× and 21g of the photobiotinylated antibody was added to the first well, serially diluted and incubated for 2 hours at room temperature or 4° C., overnight. The plate was washed 3× and 100 μL of a 1/1000 dilution of avidin peroxidase conjugate was added per well. After incubating for 1 hour at room temperature, the wells were washed 3× with washing solution. 100 μL of OPD solution (OPD buffer, o-phenylenediamine and 1 μL of 30% hydrogen peroxide per ml) were added to each well. The color development was stopped by adding 30 μL of 4N H₂SO₄ and the optical density was determined by scanning each well at 492 nm with a Fisher Scientific Multiskan RC plate reader.

Monoganglioside ELISA

GM1, GM2, GM3 and glycolylic GM3 monoganglioside were dissolved in methanol and coated overnight by drying on polystyrene microtiter plates at 0.5 μg per well. The wells were blocked with 1% BSA for 2 hours. GM2 tryptophan T15 conjugate was added to 1% BSA to a concentration of 2 μg/μl and 200 μL was added to the first row of wells and serially diluted. After incubation at room temperature for 1 hr, the wells were washed 5× with washing solution. The plate was washed 3× and 100 μL of a 1/1000 dilution of avidin peroxidase conjugate was added per well. After incubating for 1 hr at room temperature, the wells were washed 3× with washing solution. 100 μL of OPD solution (OPD buffer, o-phenylene diamine and 1 μL of 30% hydrogen peroxide/ml) were added to each well. The color development was stopped by adding 30 μL of 4N H₂SO₄ and the optical density was determined by scanning each well at 492 nm (Fisher Scientific Multiskan RC plate reader).

Photobiotinylation at Different pH

The antibodies were incubated with 100 μM biotin peptide at pHs 5, 6, 7, 8, 9, for 1 hour at room temperature and UV-crosslinked. The samples were dialyzed against PBS pH 7.4 and analyzed by capture ELISA.

Results

Screening of Biotin Amino Acids for Photo-Biotinylattion.

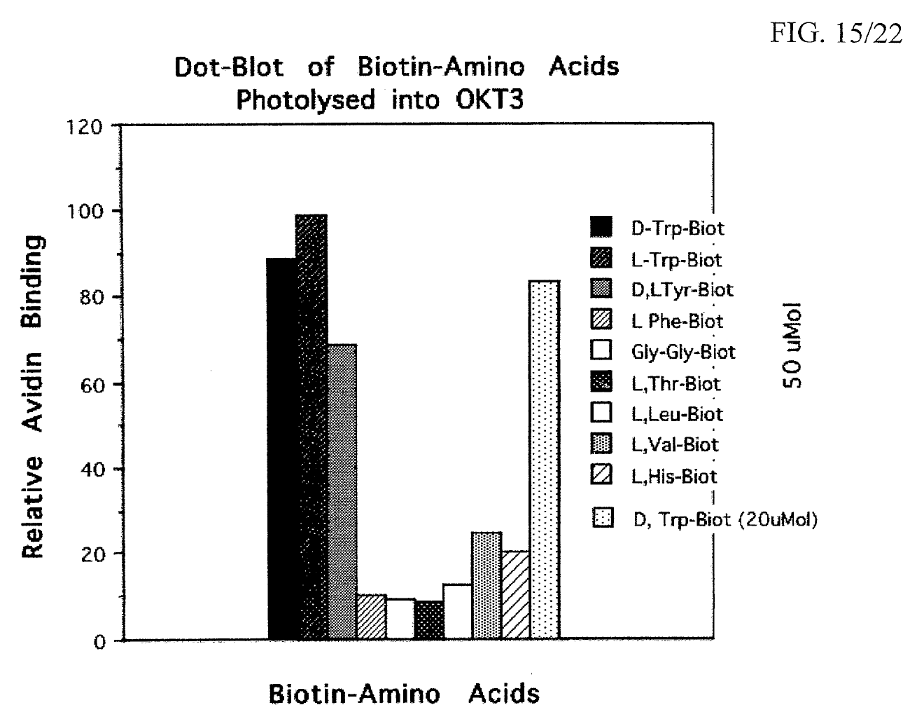

Several biotinylated amino acids were mixed with a monoclonal antibody, OKT3, and exposed to UV. The mixture was then dot-blotted and developed with avidin-HRP. The dots were scanned and the relative color intensity was recorded. As shown in FIG. 15, OKT3 photolyzed with biotinylated tryptophan yielded the strongest reaction with avidin followed by biotin-tyrosine. OKT3 photolyzed with other biotin amino acid gave only background reaction with avidin.

Titrating Trp-Biotin Photolysis.

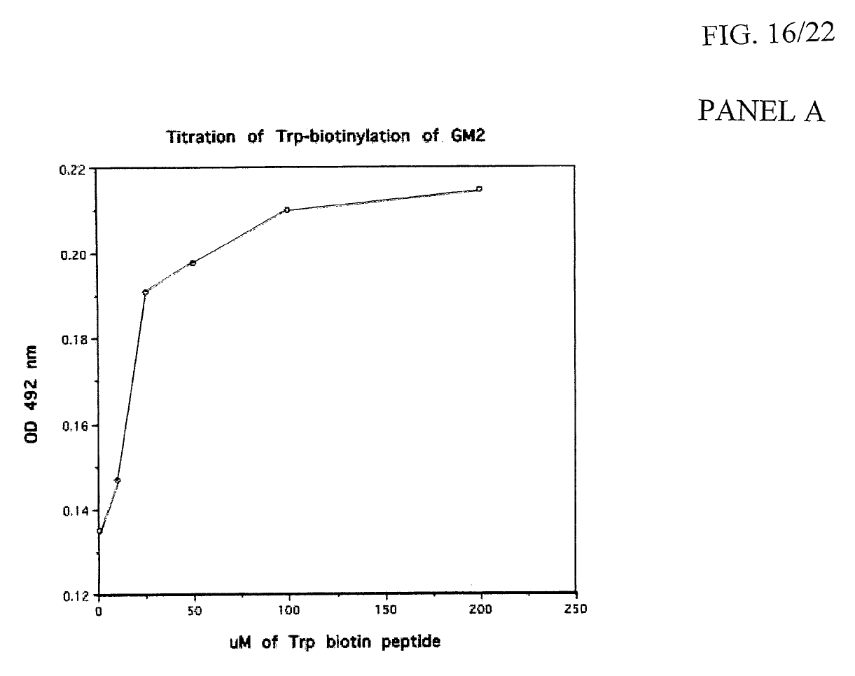

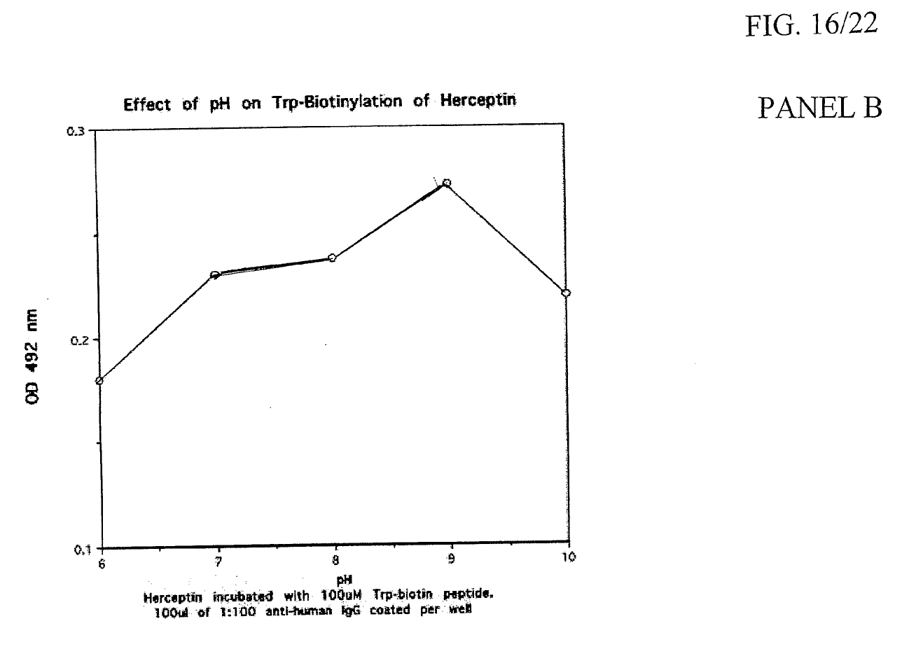

To obtain data on the affinity of biotin-Trp the monoclonal chimeric anti-ganglioside (anti-GM2) antibody was photolyzed at increasing concentrations of biotin-Trp. The results shown in FIG. 16A indicate a saturating plateau of biotinylation of the antibody at the 100 μM level. Similar results were obtained with the titration of another monoclonal chimeric antibody against ganglioside (data not shown).

The dependence of affinity Trp photobiotinylation on pH was probed. The humanized antibody Herceptin® was photolyzed at different pH. As seen in FIG. 16B, the highest biotinylation was at pH 9. Similar pH dependence on biotinylation was observed with other monoclonal antibodies (data not shown).

Testing the Covalent Attachment of the Biotin-Trp-Peptides.

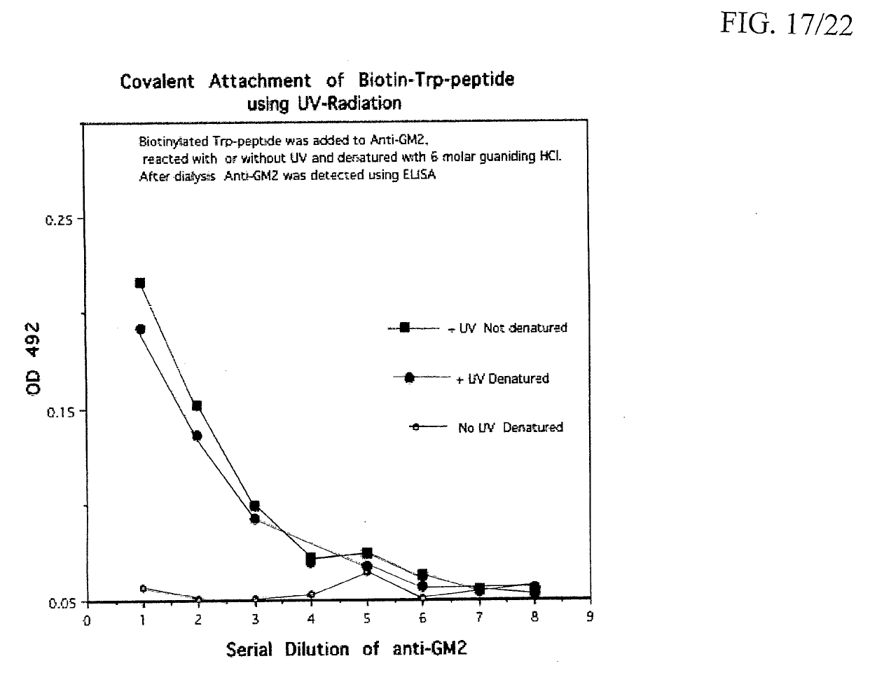

To prove that the photobiotinylation creates covalent bonds between the biotin peptide and the antibody, the biotinylated chimeric anti-ganglioside antibody was exposed to 6M guanidine HCL, then dialyzed against PBS and tested in direct avidin-HRP ELISA. FIG. 17 shows the ELISA reading of the native biotinylated anti-GM2 antibody and the de/re-natured antibody. Both preparations gave identical ELISA colors. Anti-GM2 not exposed to UV did not react with avidin in the ELISA. These results provide evidence that the photobiotinylation using a Trp-biotin peptide attaches the biotin-peptide covalently to the antibody.

Antigen Binding of Single and Multiple Biotinylated Antibodies.

Next, the use of biotin-peptides that contain terminal Trp was examined. Two kinds of Trp-biotin peptides were synthesized: 1) KAAGW containing a biotin molecule on the alpha amino group [single biotin-peptide] and 2) KAAKGEAKAAGW containing biotin molecules on the alpha and epsilon amino groups of lysine [multiple biotin-peptide].

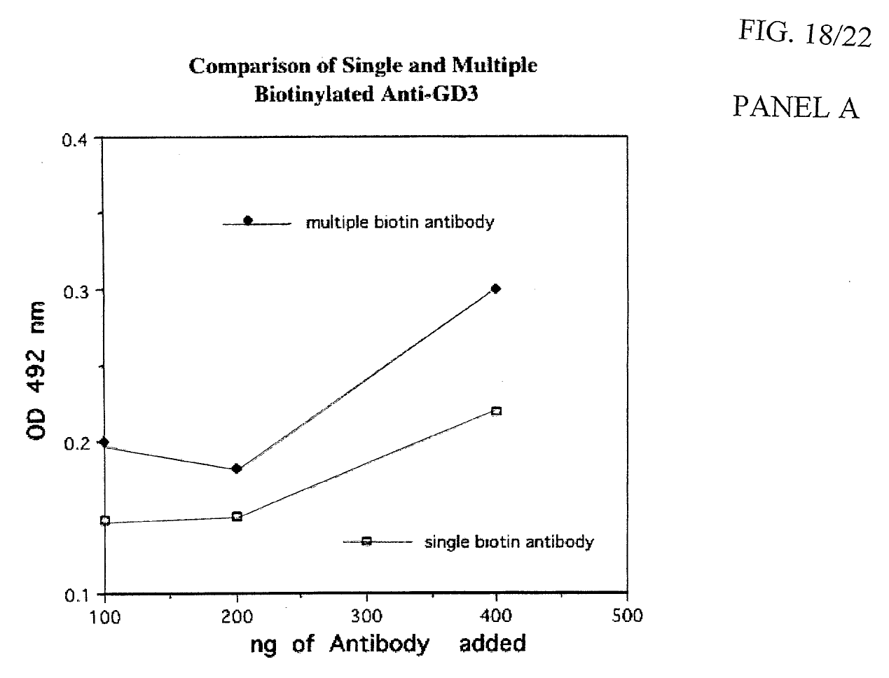

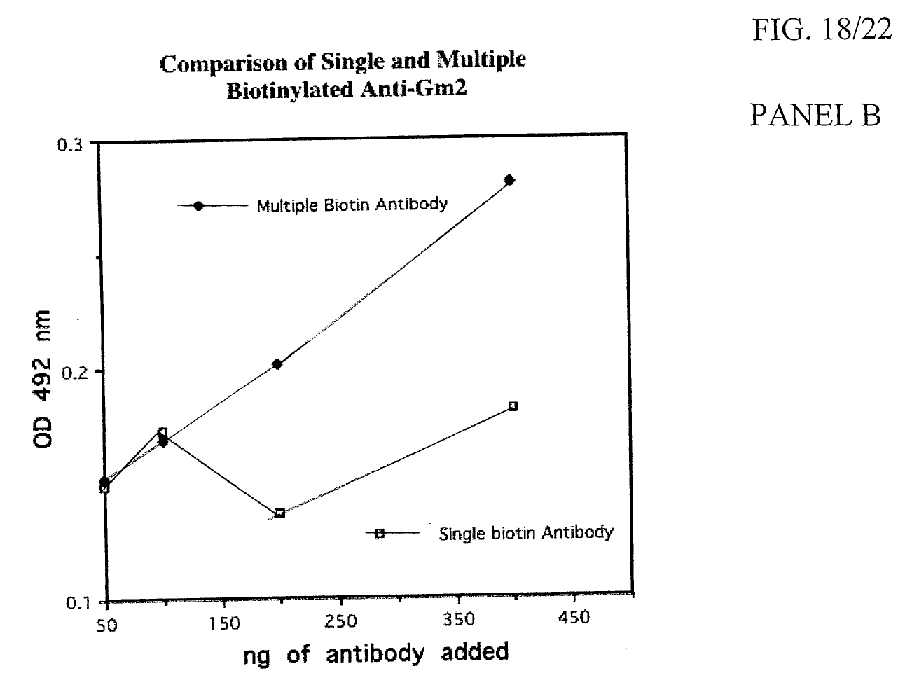

In FIG. 18A, the single biotin-peptide humanized anti-GM3 was compared to insolubilized ganglioside with the multiple biotin-peptide anti-GM3. The multiple biotin antibody produced stronger ELSIA signals with avidin-HRP. Similar differences (FIG. 18B) between a single and the multiple biotinylated antibody were seen with the chimeric anti-GM2.

Comparing the Efficiency of Photo-Biotinylation with Chemical Biotinylation.

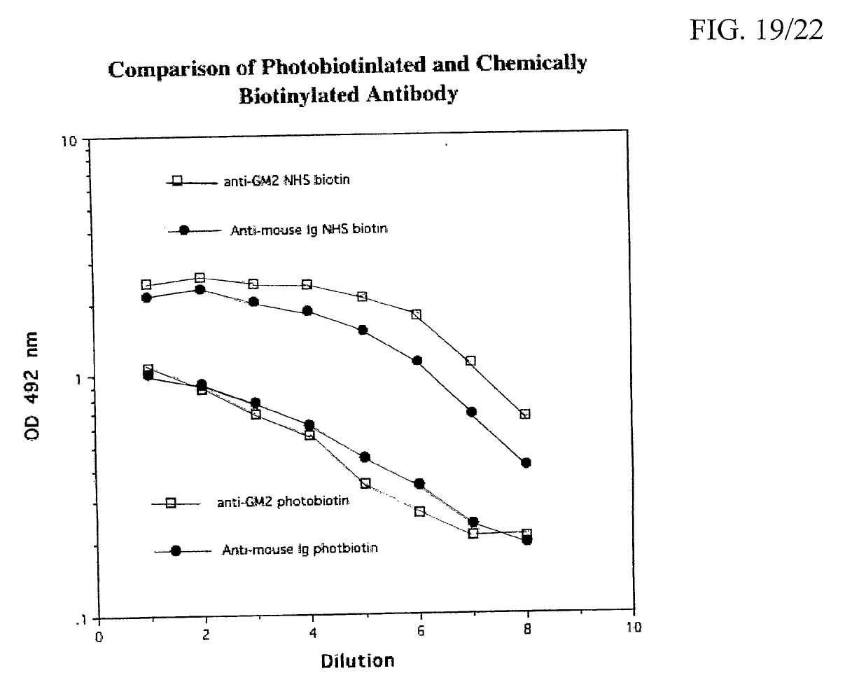

Chemical biotinylation techniques are based on the variable availability of reactive amino acid side chains to produce mixtures of biotin proteins. For antibodies the number of biotins attached is 8-12 per molecule. In contrast, affinity-based biotinylation is limited by the number of affinity sites per antibody. In targeting the nucleotide site two affinity sites are available per Ig molecule. The number of Trp sites is variable in antibodies between 3 and 5 per molecule as estimated by a commercial biotin determination assay (data not shown). In FIG. 19, the reaction of avidin-HRP with insolubilized antibodies is shown. As expected, the chemically biotinylated antibodies produce stronger ELISA readings than the photo-biotinylated antibodies.

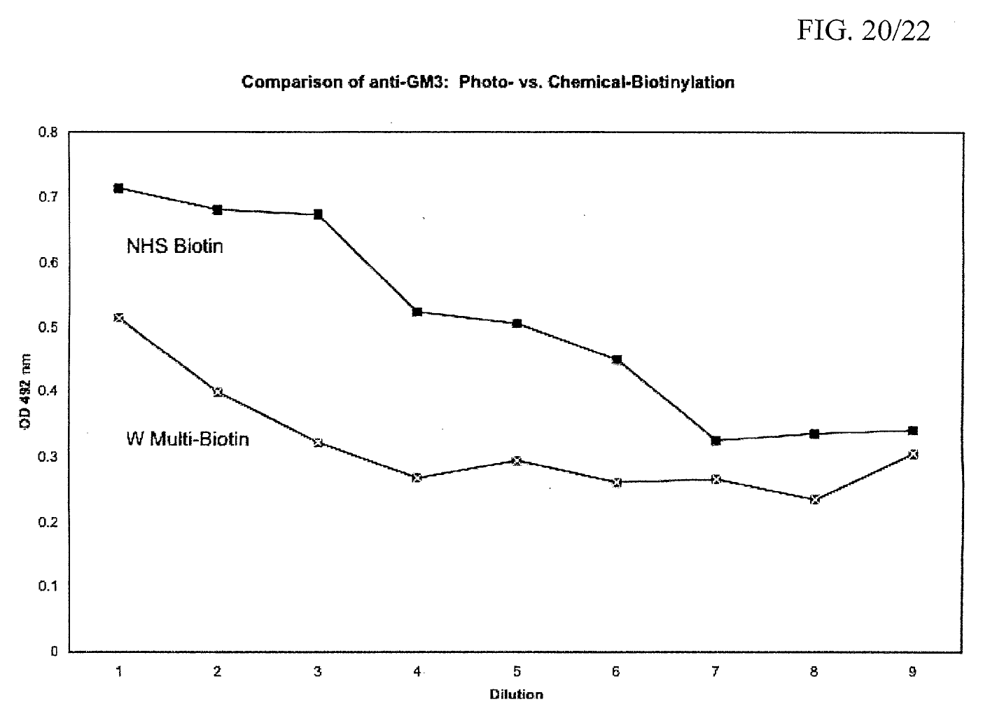

To compare the detection sensitivity in an antigen-specific ELISA, photo- and chemical biotinylation of the chimeric anti-glycolylic GM3 antibody was performed. As shown in FIG. 20, the chemically biotinylated antibody produces a stronger signal than the photo-biotinylated antibody due to the greater number of biotin molecules on the antibody with chemical method.

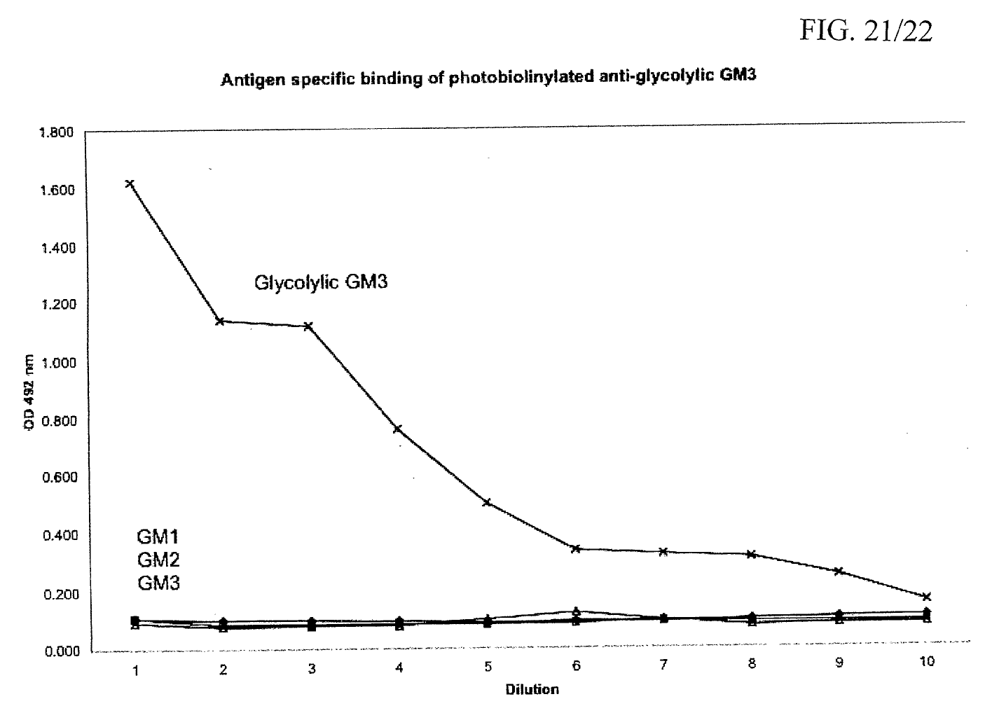

To demonstrate the antigen specificity of affinity-photobiotinylated antibody, the chimeric anti-glycolylic GM3 antibody in ELISA was used. As seen in FIG. 21, the photo-biotin antibody recognizes its target antigen, not control ganglioside GM1, GM2 and GM2.

Discussion

Conjugating peptides with biological or chemical properties is an attractive method to enhance the potency of antibodies or endow antibodies with diagnostic and therapeutic utility [Zhao, et al (2001); Zhao, et al (2002)a; Zhao, et al (2002)b]. For example, the targeting of antibodies has been increased by conjugating autophilic peptides to produce dimerizing antibodies with enhanced targeting and induction of apoptosis. In another study, membrane transporting sequence (MTS) was conjugated to antibodies and demonstrated that such MTS-antibodies penetrate the cellular membranes of living cells without harming the cells [Zhao, et al (2001)]. MTS antibodies against caspase-3 enzyme can inhibit induction of apoptosis in tumor cells. Attaching a peptide from the C3d complement fragment enhances the immune response to antibody vaccines creating a molecular adjuvant vaccine [Lou (1998)].

In all of these conjugations the invariant carbohydrate or the invariant nucleotide binding site were used. Both methods have drawbacks involving complex chemical reactions. The carbohydrate method requires oxidation of the antibody to create a reactive aldehyde and the nucleotide affinity photocrosslinking involves the synthesis of an azido-adenosine peptide [Lou and Kohler (1998)].

Here is presented a simple one-step affinity crosslinking technique for peptides based on the discovery that antibodies can be photo-crosslinked to aromatic hydrocarbon moieties (AHMs), including heterocyclic amino acids, such as tryptophan. Thus, peptides that contain terminal tryptophan are affinity photo-crosslinking reagents for antibodies.

These new affinity conjugation methods have been demonstrated using biotinylated peptides. Exposing UV energy to a mixture of antibody and Trp-biotin peptides produces a biotin antibody that can be used in ELISA and other biotin-based detection methods. Such affinity-biotinylated antibodies have a defined number of biotins attached that are less than conventional biotinylation chemistries, but sufficient to produce useful signals in ELISA. Currently, the Trp-affinity photo-crosslinking method is used to attach peptides with biological and chemical properties similar to those previously published [Lou et al. (1998); Zhao, et al (2001); Zhao, et al (2002)a; Zhao, et al (2002)b].

Advantages of the tryptophan affinity-site based biotinylation are: (i) gentle one-step procedure without modifying amino acid side chains, and (ii) generates a reproducible antibody product labeled with defined number of biotin molecules.

### Example 10

### Detection of Circulating Ox-Ldl with Super-Antibodies

The ability of autophilic antibodies, prepared according to the principles of the present invention, to recognize epitopes of circulating ox-LDL can be determined by conducting a sandwich assay. First, goat anti-mouse IgG-Fc antiserum is coated on microtiter wells, to which mouse mAbs having specific binding affinity for LDL particles, such as for apoB, are added. Next, plasma is contacted with the coated microtiter wells, followed by extensive washing. Then, a super-antibody, comprising a mAb specific for ox-LDL conjugated to an autophilic peptide is added to top the sandwich. The completed sandwich can be visualized by a labeled secondary antibody specific for the autophilic peptide. Super-antibodies having specific binding affinity for ox-LDL should show at least a several-fold increase in detection over analogous super-antibodies nonspecific for ox-LDL. Controls for ox-LDL can be provided by Cu+2-oxidized LDL (see U.S. Pat. No. 6,225,070 to Witztum et al.).

### Example 11

### Inhibition of Chronic Inflammation in Atherosclerosis

Chronic inflammation leading to atherosclerosis can be inhibited by the capacity of super-antibodies to bind avidly to ox-LDL, thereby blocking or reducing uptake of ox-LDL by macrophages. Humanized autophilic antibodies having specificity for ox-LDL are administered to a patient according to the regimen described hereinabove. The self-binding property of the autophilic antibodies increases their affinity for ox-LDL over that of unconjugated antibodies, and reduces recognition of the LDL particles by macrophages. Macrophage binding to ox-LDL should be effectively inhibited greater than 50% in the presence of the immunoconjugate.

As will be apparent to those skilled in the art, certain improvements and modifications are possible in the practice of this invention based on the foregoing disclosure, without departing from the spirit or scope thereof. Accordingly, the scope of the invention is defined by the claims appended hereto and equivalents thereof.

### REFERENCES

The pertinent disclosures of the following references are incorporated herein by reference:
1. Award, M., et al. “Modification of monoclonal antibody carbohydrates by oxidation, conjugation or deoxymannojirmicin does not interfere with antibody effector functions,” Cancer Immunol, Immunother., 1994, 38: 23.
2. Binder J., et al. “Pneumococcal vaccination decreases atherosclerotic lesions formation: molecular mimicry between Streptococcus pneumoniae and oxidized LDL,” Nature Medicine, 2003, 195: 771.
3. Caldas C., et al., “Humanization of the anti-CD18 antibody 6.7: an unexpected effect of a framework residue in binding to antigen,” Mol. Immunol., 2003, 39: 941-952.
4. Chapman P., et al., “Mapping effector functions of a monoclonal antibody to GD3 by characterization of a mouse-human chimeric antibody,” Cancer Immunol. Immunother., 1994,39(3): 198-204.
5. Dean G., et al., “Peptide mapping of feline immunodeficiency virus by IFN-gamma ELISPOT,” Vet. Immunol. Immunopathol., 2004, 100: 49-59.
6. Ghetie M., et al., “Anti-CD19 inhibits the growth of human B-cell tumor lines in vitro and of Daudi cells in SCID mice by inducing cell cycle arrest,” Blood, 1994, 83:1329-1336.
7. Ghetie M., et al., “Homodimerization of tumor-reactive monoclonal antibodies markedly increases their ability to induce growth arrest or apoptosis of tumor cells,” Proc. Natl. Acad. Sci. USA, 1997, 94: 7509-7514.
8. Gonzales N., et al., “SDR grafting of a murine antibody using multiple human gemmline templates to minimize its immunogenicity,” Mol. Immunol., 2004, 41(9): 863-872.
9. Hakenberg J., et al., “MAPPP: MHC class I antigenic peptide processing prediction,” Appl. Bioinformiatics, 2003, 2(3):155-158.
10. Halpern R. et al “Human anti-phosphorylcholine antibodies share idiotopes and are self-binding” J. Clin. Invest., 1991, 88: 476-482.
11. Hasbold J., et al., “Anti-immunoglobulin antibodies induce apoptosis in immature B cell lymphomas,” Eur. J. Immunol., 1990, 20: 1685-1690.
12. Horkko, S. et al. “Immunological response to axidized LDL,” Free Dadic. Biol Med., 2000, 28: 1771.
13. Isaacs J., et al., “Helplessness as a strategy for avoiding antiglobulin responses to therapeutic monoclonal antibodies,” Ther. Immunol, 1994, 1: 303-312.
14. Kang, C-Y, et al., “Immunoglobulin with complementary paratope and idiotope,” J. Exp. Med., 1986, 163: 787.
15. Kang C—Y, et al. “Inhibition of self-binding antibodies (autobodies) by a VH-derived peptide,” Science, 1988, 240:1034-1036.
16. Kaveri S., et al., “Self-binding antibodies (autobodies) form specific complexes in solution,” J. Immunol., 1990, 145: 2533-2538.
17. Kaveri S., et al., “Antibodies of different specificities are self-binding: implication for antibody diversity,” Mol. Immunol., 1991, 2: 733-778.
18. Kohler H., et al., “Superantibody activities: new players in innate and adaptive immune responses,” Immunol. Today, 1998, 19: 221-227.
19. Kohler, H, et al. “Superantibody activities: new players in innate and adaptive immune responses,” Immunol. Today, 1998, 19: 221.
20. Kohler H., “Superantibodies: synergy of innate and acquired immunity,” Appl. Biochem. Biotechnol., 2000, 83: 1-9.
21. Leger O., et al., “Humanization of a mouse antibody against human alpha-4 integrin: a potential therapeutic for the treatment of multiple sclerosis,” Mol. Immunol., 2003, 39: 941-952.
22. Libby, P., et al. “Inflammation and atherosclerosis,” Circulation, 2002, 105: 1135-1141.
23. Lieberman, R. et al. “Genetics of a new IgVH (T15 idiotype) marker in the mouse regulating natural antibody to phosphorylcholine,” J Exp Med, 1974, 139: 983-10001.
24. Lin Y., et al., “Inhibition of nuclear translocation of transcription factor NF-KB by a synthetic peptide containing a cell membrane-permeable motif and nuclear localization sequence,” J Biol Chem., 1995, 270: 14255-14258.
25. Lou, D., et al., “Enhanced molecular mimicry of CEA using photoaffinity crosslinked C3d peptide,” Nat. Biotechnol., 1998, 16: 458.
26. Miles M., et al., “Multiple peptide synthesis (Pepscan method) for the systematic analysis of B- and T-cell epitopes: Application to parasite proteins,” Parasitol. Today, 1989, 5: 397-400.
27. Miles, E., et al., “Photoactivation and photoaffinity labeling of tryptophan synthetase+□²□, complex by the product analogue 6-azido-L-tryptophan,” Biochemistry, 1985, 24: 4694.
28. Pavlinkova, G., et al., “Site-specific photobiotinylation of immunoglobulins, fragments and light chain dimmers,” J. Immunol. Methods, 1997, 201: 77.
29. Rajagopalan, K., et al., “Novel unconventional binding site in the variable domain of immunoglobulins,” PNAS USA, 1996, 93: 6019.
30. Roque-Navarro L. et al., “Humanization of predicted T-cell epitopes reduces the immunogenicity of chimeric antibodies: new evidence supporting a simple method.” Hybrid Hybridomics, 2003, 22: 245-257.
31. Schellekens H., “Immunogenicity of therapeutic proteins: clinical implications and future prospects,” Clin Ther., 2002, 24:1720-1740.
32. Shaw, P., et al., “Natural antibodies with the T15 idiotype may act in atherosclerosis, apoptotic clearance, and protective immunity,” J. Clin. Invest., 2000, 1731.
33. Suzuki Y., et al., “Aberrant expression of ganglioside and asialoglyco-sphingolipid antigens in adult T-cell leukemia cells,” Jpn J Cancer Res., 1987, 78: 1112-1120.
34. Veeraraghavan S. et al., “Mapping of the immunodominant T cell epitopes of the protein topoisomerase I,” Ann Rheum Dis., 2004, 63: 982-987.

35. Wallen-Ohman M., et al., “Antibody-induced apoptosis in a human leukemia cell line is energy dependent: thermochemical analysis of cellular metabolism,” Cancer Letters, 1993, 75: 103-109.
36. Ward R., et al., “Regulation of an idiotype+ B cell lymphoma. Effects of antigen and anti-idiotypic antibodies on proliferation and Ig secretion,” J. Immunol., 1988, 141: 340-345.
37. Zhao Y., et al., “Enhanced Anti-B-cell Tumor Effects with Anti-CD20 Superantibody,” J. Immunotherapy, 2002a, 25: 57-62.
38. Zhao, Y., et al., “Enhancing Tumor Targeting and Apoptosis Using Non-Covalent Antibody Homo-dimers”, J. Immunotherapy, 2002b, 25: 396-404.
39. Zhao Y., et al., “MTS-Conjugated-Antiactive caspase-3 antibodies Inhibit Actinomycin D-induced apoptosis,” Apoptosis 2003, 8: 631-637.

What is claimed is:

1. A method of covalently linking a photoactivatable compound to an immunoglobulin, comprising:
(a) forming an admixture of the photoactivatable compound and the immuno-globulin, which has a binding affinity for the photoactivatable compound; and
(b) subjecting the admixture to photoactivation conditions effective to covalently link the photoactivatable compound to the immunoglobulin,
wherein the photoactivatable compound contains at least one aromatic hydrocarbon moiety and does not contain an azido, purine or pyrimidine group.

2. The method of claim 1, wherein the photoactivable compound comprises a peptide having self-binding, membrane-penetrating, adjuvant, and/or enzymatic properties.

3. The method of claim 2, wherein the photoactivable compound comprises a peptide containing from 5 to 30 amino acid residues.

4. The method of claim 2, wherein the peptide contains an amino acid sequence selected from SEQ ID NO: 1, SEQ ID NO. 2, SEQ ID NO: 4, SEQ ID NO: 5, SEQ ID NO: 6, SEQ ID NO: 7, SEQ ID NO: 10, SEQ ID NO: 11, SEQ ID NO: 12 and SEQ ID NO: 13.

5. The method of claim 2, wherein said aromatic hydrocarbon moiety is located at a terminal position of the peptide, or in an internal position.

6. The method of claim 1, wherein the immunoglobulin is a polyclonal antibody, monoclonal antibody, Fab fragment, or F(ab′)₂ fragment.

7. The method of claim 1, wherein said binding affinity occurs at an affinity site located in a variable domain of the immunoglobulin.

8. The method of claim 1, wherein said binding affinity is demonstrable by competitive binding with an aromatic reporter molecule.

9. The method of claim 1, wherein a plurality of said photoactivatable compounds are covalently linked to the immunoglobulin.

10. The method of claim 1, wherein the aromatic hydrocarbon moiety comprises at least one aryl, polynuclear aryl, heterocycle, or polynuclear heterocycle.

11. The method of claim 1, wherein the aromatic hydrocarbon moiety comprises a benzene, naphthalene, anthracene, phenanthrene, pyrrole, furan, thiophene, imidazole, pyrazole, oxazole, thiazole, pyridine, indole, benzofuran, thionaphthene, quinoline, or isoquinoline group.

12. The method of claim 1, wherein the aromatic hydrocarbon moiety comprises an amino acid residue selected from tryptophan, tyrosine, histidine, and phenylalanine.

13. The method of claim 1, wherein the immunoglobulin has specific binding affinity for a cancer-related antigen, a caspase enzyme, ox-LDL, or cellular receptor.

14. An immunoconjugate formed by the method of claim 1.

15. The immunoconjugate of claim 14, which has autophilic, membrane-penetrating, adjuvant, and/or enzymatic properties.

16. An immunoconjugate comprising an immunoglobulin covalently linked to at least one peptide, which immunoconjugate does not contain an azido, purine or pyrimidine group.

17. The immunoconjugate of claim 16, wherein the immunoglobulin is a polyclonal antibody, monoclonal antibody, Fab fragment, or F(ab′)₂ fragment.

18. The immunoconjugate of claim 16, wherein the peptide contains from 5 to 30 amino acid residues.

19. The immunoconjugate of claim 16, wherein the peptide has self-binding, membrane-penetrating, adjuvant, and/or enzymatic properties.

20. The immunoconjugate of claim 16, wherein the peptide contains an autophilic amino acid sequence selected from the group consisting of SEQ ID NO: 1, SEQ ID NO: 4, SEQ ID NO: 5, SEQ ID NO: 6, SEQ ID NO: 10 and SEQ ID NO: 11.

21. The immunoconjugate of claim 16, wherein the peptide contains a membrane-penetrating amino acid sequence selected from the group consisting of SEQ ID NO. 2, SEQ ID NO: 7, SEQ ID NO. 12 and SEQ ID NO: 13.

22. The immunoconjugate of claim 16, wherein the immunoglobulin and peptide are joined by a photoactivated aromatic hydrocarbon moiety.

23. The immunoconjugate of claim 22, wherein the photoactivated aromatic hydrocarbon moiety is located at a terminal position of the peptide.

23. The immunoconjugate of claim 22, wherein said aromatic hydrocarbon moiety comprises at least one aryl, polynuclear aryl, heterocycle, or polynuclear heterocycle.

24. The immunoconjugate of claim 23, wherein the aromatic hydrocarbon moiety comprises a benzene, naphthalene, anthracene, phenanthrene, pyrrole, furan, thiophene, imidazole, pyrazole, oxazole, thiazole, pyridine, indole, benzofuran, thionaphthene, quinoline, or isoquinoline group.

25. The immunoconjugate of claim 24, wherein the aromatic hydrocarbon moiety comprises an amino acid residue selected from tryptophan, tyrosine, histidine, and phenylalanine.

26. The immunoconjugate of claim 16, wherein the immunoglobulin has specific binding affinity for a cancer-related antigen, a caspase enzyme, ox-LDL, or cellular receptor.

27. A composition comprising a pharmacologically effective amount of the immunoconjugate of claim 16 and a pharmaceutically acceptable carrier.

28. A method of preventing or treating atherosclerosis in a patient comprising administering to the patient an immunoconjugate having specific binding affinity for oxidized low density lipoprotein (ox-LDL) and autophilic properties, at a dose effective to block or reduce uptake of ox-LDL by macrophages, thereby inhibiting chronic inflammation associated with atherosclerosis.

29. The method of claim 28, wherein the immunoconjugate binds phosphorylcholine and/or expresses T15 idiotype.

30. The method of claim 28, wherein the immunoconjugate is humanized.

31. The method of claim 28, wherein the immunoconjugate contains an amino acid sequence selected from the group consisting of SEQ ID NO: 1, SEQ ID NO: 4, SEQ ID NO: 5, SEQ ID NO: 6, SEQ ID NO: 10, and SEQ ID NO: 11.

32. The method of claim 28, wherein a predetermined initial dose of the immunoconjugate, and a predetermined later dose, are administered to the patient.

33. The method of claim 28, wherein a maintenance dose of the immunoconjugate is administered to the patient.

34. A method of detecting atherosclerotic plaques in a patient's vascular system, comprising:
(a) administering to the patient an immunoconjugate, which immunoconjugate has a specific binding affinity for oxidized low density lipoprotein (ox-LDL) and autophilic properties; and
(b) determining sites of immunoconjugate concentration in the patient's vascular system, thereby detecting the atherosclerotic plaques.

35. The method of claim 34, wherein the immunoconjugate binds phosphorylcholine and/or expresses T15 idiotype.

36. The method of claim 34, wherein the immunoconjugate is humanized.

37. The method of claim 34, wherein the immunoconjugate comprises an autophilic peptide containing an amino acid sequence selected from the group consisting of SEQ ID NO: 1, SEQ ID NO:4, SEQ ID NO: 5, SEQ ID NO: 6, SEQ ID NO: 10, and SEQ ID NO: 11.

38. A method of detecting a cell undergoing apoptosis, comprising:
(a) contacting the cell with an immunoconjugate comprised of an immunoglobulin conjugated to an autophilic peptide, wherein the immunoconjugate specifically binds to an antigenic determinant of a cell undergoing apoptosis; and
(b) detecting the presence or absence of the immunoconjugate bound to the cell.

39. The method of claim 38, wherein the antigenic determinant comprises membrane phosphorylcholine or phosphatidylserine.

40. The method of claim 38, wherein the autophilic peptide comprises an amino acid sequence selected from the group consisting of SEQ ID NO: 1, SEQ ID NO: 4, SEQ ID NO: 5, SEQ ID NO: 6, SEQ ID NO: 10, and SEQ ID NO: 11.

41. The method of claim 38, wherein said detecting employs flow cytometry, fluorescent microscopy, histological staining, or in vivo imaging.

42. The method of claim 38, wherein the immunoconjugate is labeled with fluorescein and the fluorescein label is detected.
